SEQUENCE LISTING (25 sequences)

SEQ ID NO: 1 - DNA, 1356 bp, Mus musculus
  5′UTR (1)..(10)
  3′UTR (725)..(1356)
tggccgcgggatggcggctctgagaagttgggtgactcggagcgtctgctctctgttcaggtacagacagcgttttcctgtcttggctaactctaagaaacgctgtttctcagaattgataaaaccatggcacaaaactgtgttgactggatttggcatgacactgtgtgcggttcctattgctcagaaatcggagcctcattctctcagtaacgaagcattgatgaggagggctgtgtctttggtaacagatagcacctctacctttctgtctcaaaccacctacgcgctgattgaagcaatcaccgagtatactaaggctgtttacacgttagtgtctctgtaccgacaatatacaagtttacttgggaagatgaattcccaggaggaagatgaggtgtggcaggtaattataggagccagagttgagatgacttcaaaacagcaggaatatttgaagttggagaccacttggatgacagcggttggcctttcagagatggctgctgaggctgcctatcaaactggagcagatcaggcctccataactgccaggaatcacatccagttggtgaagtcacaggtacaggaggtgcgccagctctcccagaaggcagaaaccaagttggctgaagcacagacaaaagagctgcaccagaaagcacaggaagtgagtgatgagggggctgaccaggaagaggaggcctacctgcgtgaagattgagggcctgagcccagtgccctgtccatccactctgtggggaaagcgggtggatggtatccacctaatgttgtcccagctctgcagagctaacagtcctgccattgacagtcagaccagagccttctgtgagctgtgcctggccccttgtctgctcacaccctctgtgcctggttcttagctctgggcgtggctcagcactgtccctcagcactgctcctctcttgctgatcttttagctcacacccaaagcatttcaccaacctgggccaacagaggaggggcctttcctgccacacccttaagttcagtagctgtttaactacagtttttaatgttactcggatgttcaagtgatttagtgcttgcttctccacagggaagtggggggcttgtctggtttgtaacatttctgtgtttataatgtcctttctccctgtgagcacagctcagctatggagttactcagggcgtgaacagctcacctggtgctccccacaggcccttctctaacacgtacttcttgtttgttccttctttgaatttctctgctgtatccaaaggaagagaagtttagtgtttgcattaaaaaagagagattgggaaaaaaaaaaaaaaaaaaaaaaaaaaaaaaa

SEQ ID NO: 2 - protein, 237 aa, Mus musculus
MAALRSWVTRSVCSLFRYRQRFPVLANSKKRCFSELIKPWHKTVLTGFGMTLCAVPIAQKSEPHSLSNEALMRRAVSLVTDSTSTFLSQTTYALIEAITEYTKAVYTLVSLYRQYTSLLGKMNSQEEDEVWQVIIGARVEMTSKQQEYLKLETTWMTAVGLSEMAAEAAYQTGADQASITARNHIQLVKSQVQEVRQLSQKAETKLAEAQTKELHQKAQEVSDEGADQEEEAYLRED

SEQ ID NO: 3 - DNA, 714 bp, Mus musculus
atggcggctctgagaagttgggtgactcggagcgtctgctctctgttcaggtacagacagcgttttcctgtcttggctaactctaagaaacgctgtttctcagaattgataaaaccatggcacaaaactgtgttgactggatttggcatgacactgtgtgcggttcctattgctcagaaatcggagcctcattctctcagtaacgaagcattgatgaggagggctgtgtctttggtaacagatagcacctctacctttctgtctcaaaccacctacgcgctgattgaagcaatcaccgagtatactaaggctgtttacacgttagtgtctctgtaccgacaatatacaagtttacttgggaagatgaattcccaggaggaagatgaggtgtggcaggtaattataggagccagagttgagatgacttcaaaacagcaggaatatttgaagttggagaccacttggatgacagcggttggcctttcagagatggctgctgaggctgcctatcaaactggagcagatcaggcctccataactgccaggaatcacatccagttggtgaagtcacaggtacaggaggtgcgccagctctcccagaaggcagaaaccaagttggctgaagcacagacaaaagagctgcaccagaaagcacaggaagtgagtgatgagggggctgaccaggaagaggaggcctacctgcgtgaagattga

SEQ ID NO: 4 - protein, 237 aa, Mus musculus; identical to SEQ ID NO: 2

SEQ ID NO: 5 - DNA, 534 bp, Mus musculus
tcggagcctcattctctcagtaacgaagcattgatgaggagggctgtgtctttggtaacagatagcacctctacctttctgtctcaaaccacctacgcgctgattgaagcaatcaccgagtatactaaggctgtttacacgttagtgtctctgtaccgacaatatacaagtttacttgggaagatgaattcccaggaggaagatgaggtgtggcaggtaattataggagccagagttgagatgacttcaaaacagcaggaatatttgaagttggagaccacttggatgacagcggttggcctttcagagatggctgctgaggctgcctatcaaactggagcagatcaggcctccataactgccaggaatcacatccagttggtgaagtcacaggtacaggaggtgcgccagctctcccagaaggcagaaaccaagttggctgaagcacagacaaaagagctgcaccagaaagcacaggaagtgagtgatgagggggctgaccaggaagaggaggcctacctgcgtgaagattga

SEQ ID NO: 6 - protein, 177 aa, Mus musculus
SEPHSLSNEALMRRAVSLVTDSTSTFLSQTTYALIEAITEYTKAVYTLVSLYRQYTSLLGKMNSQEEDEVWQVIIGARVEMTSKQQEYLKLETTWMTAVGLSEMAAEAAYQTGADQASITARNHIQLVKSQVQEVRQLSQKAETKLAEAQTKELHQKAQEVSDEGADQEEEAYLRED

SEQ ID NO: 7 - protein, 202 aa, Homo sapiens
LIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 8 - protein, 177 aa, Homo sapiens
SEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 9 - protein, 84 aa, Rattus sp.
MAALRSWMTRSVTFLFRYGQRFPVSANSKKRCFSELIRPWHKTMLTGFGVTLCAVPIAQKSEPQSLSNEALMRRAVSLVTNSTS

SEQ ID NO: 10 - protein, 73 aa, Platichthys flesus
VQKSGEWTNAANMSIASLSVARGLFTQQVETLTHDSLIRRAVSVVTDSSSTFLSQTTLALIDALTDYSKAVHT

SEQ ID NO: 11 - protein, 7 aa, synthetic
  VARIANT (3)..(3): X = L or I
  VARIANT (4)..(4): X = Q o K
  VARIANT (5)..(5): X = L or I
NHXXXVK

SEQ ID NO: 12 - protein, 17 aa, synthetic
  VARIANT (1)..(1): X = any amino acid
  VARIANT (2)..(2): X = any amino acid
  VARIANT (11)..(11): X = Q or K
  VARIANT (13)..(13): X = L or I
  VARIANT (14)..(14): X = L or I
XXSEEEDEVWXVXXGAR

SEQ ID NO: 13 - protein, 4 aa, synthetic
  VARIANT (4)..(4): X = L or I
PHSX

SEQ ID NO: 14 - protein, 4 aa, synthetic
  VARIANT (2)..(2): X = L or I
  misc_feature (3)..(3): M is methionine sulfoxide
AXMR

SEQ ID NO: 15 - protein, 11 aa, synthetic
  VARIANT (1)..(1): X = Q or K
  VARIANT (2)..(2): X = L or I
  VARIANT (8)..(8): X = Q or K
  VARIANT (9)..(9): X = L or I
XXVEEVHXXSR

SEQ ID NO: 16 - protein, 5 aa, synthetic
  VARIANT (4)..(4): X = L or I
PHSXS

SEQ ID NO: 17 - protein, 13 aa, synthetic
  VARIANT (7)..(7): X = Q or K
  VARIANT (12)..(12): X = Q or K
AAEAAYXTGADXA

SEQ ID NO: 18 - protein, 12 aa, synthetic
  VARIANT (1)..(1): X = L or I
  VARIANT (3)..(3): X = Q or K
  VARIANT (8)..(8): X = L or I
  VARIANT (9)..(9): X = L or I
  VARIANT (12)..(12): X = L or I
XSXTTYAXXEAX

SEQ ID NO: 19 - protein, 7 aa, synthetic
NHIQLVK

SEQ ID NO: 20 - protein, 13 aa, synthetic
  misc_feature (12)..(12): M is methionine sulfoxide
SEPHSLSSEALMR

SEQ ID NO: 21 - protein, 11 aa, synthetic
LQVEEVHQLSR

SEQ ID NO: 22 - protein, 13 aa, synthetic
SEPHSLSSEALMR

SEQ ID NO: 23 - protein, 17 aa, synthetic
  misc_feature (1)..(1): M is methionine sulfoxide
MNSEEEDEVWQVIIGAR

SEQ ID NO: 24 - protein, 32 aa, synthetic
  misc_feature (6)..(6): M is methionine sulfoxide
  misc_feature (14)..(14): M is methionine sulfoxide
LETTWMTAVGLSEMAAEAAYQTGADQASITAR

SEQ ID NO: 25 - protein, 29 aa, synthetic
AVSLVTDSTSTFLSQTTYALIEAITEYTK

(19) United States
(12) Patent Application Publication
(10) Pub. No.: US 2002/0110851 A1
(43) Pub. Date: Aug. 15, 2002

(54) Novel polypeptides, modulatory agents therefor and methods of using them

(75) Inventors: Anne Marie Verhagen, Northcote (AU); Paul Gerald Ekert, Elsternwick (AU); David Lawrence Vaux, Fairfield (AU)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: The Walter and Eliza Hall Institute of Medical Research of Royal Parade

(21) Appl. No.: 09/798,116

(22) Filed: Mar. 2, 2001

(30) Foreign Application Priority Data
Mar. 2, 2000 (AU) PQ5995

(51) Int. Cl. C12P 21/02; C12N 5/06; C07H 21/04

(52) U.S. Cl. 435/69.1; 435/325; 435/320.1; 536/23.2

(57) ABSTRACT

A pro-apoptotic polypeptide, designated DIABLO, is disclosed which inhibits the activity of IAPs, including animal and viral IAPs. Also disclosed are methods of using DIABLO polypeptides and DIABLO-encoding polynucleotides to screen for modulatory agents that modulate the level and/or functional activity of DIABLO, as well as methods for detecting cell death or apoptosis, and for diagnosis of conditions relating to the expression or activation of DIABLO. The invention also discloses compositions for treating and/or preventing such DIABLO-related conditions.

### FIELD OF THE INVENTION

THE INVENTION relates in general to agents that modulate cell death. More particularly, the present invention relates to novel polypeptides that promote cell death, and to polynucleotides encoding these polypeptides. The invention also relates to methods for detecting cell death and to methods of diagnosing a condition associated with expression or activation of the aforementioned cell death-promoting polypeptides. The invention also relates to methods of screening for modulatory agents that modulate the level and/or functional activity of these polypeptides and to the use of those agents in compositions for treating and/or preventing conditions associated with the expression or activation of these polypeptides.

### BACKGROUND OF THE INVENTION

Apoptosis is a highly conserved process by which metazoan organisms remove unwanted cells (Vaux and Korsmeyer, 1999). The key effector enzymes of apoptosis are a family of cysteine proteases termed caspases (Alnemri et al., 1996). Upstream caspases are activated by adaptor proteins such as FADD and Apaf-1, which bind to the pro-domains of the caspase zymogens (Muzio et al., 1996, Boldin et al., 1996, Zou et al, 1997). The ability of the adaptor molecules to activate the caspases can be controlled by several families of regulatory molecules. For example, apoptosis signalled via Apaf-1 can be inhibited by anti-apoptotic members of the Bcl-2 family (Pan et al., 1998, Hu et al., 1998), and apoptosis signalled by FADD can be inhibited by FLIP (Irmler et al., 1997).

Experiments in insect and mammalian systems have shown that caspase-dependent apoptosis can also be regulated by members of the inhibitor of apoptosis (IAP) protein family. IAPs were first identified as baculoviral gene products that inhibited the defensive apoptotic response of insect cells following infection (Crook et al., 1993). Subsequently, IAP homologues have been identified in both invertebrates and vertebrates (Rothe et al., 1995, Uren et al., 1996, Liston et al., 1996, Duckett et al., 1996).

DIAP1 from Drosophila melanogaster can bind to and inhibit Drosophila caspases DCP-1, DRICE and DRONC (Hawkins et al., 1999, Kaiser et al., 1998, Dorstyn et al., 1999). Inhibition of apoptosis by DIAP1 can be countered by insect pro-apoptotic signalling molecules Grim, Reaper and HID which appear to promote caspase activation by binding directly to DIAP1, and thereby preventing it from inhibiting caspase activity (Hay et al., 1995, Wang et al., 1999, Kaiser et al., 1998). While no mammalian homologues of Grim, Reaper or HID have been identified, because baculoviral and insect IAPs are able to inhibit apoptosis in mammalian cells, their mechanism of action is likely to be conserved (Hawkins et al., 1996, Hay et al., 1995).

The mammalian IAP homologue A (MIHA, XIAP, h-ILP) inhibits apoptosis induced by chemotherapeutic drugs or UV radiation, and expression of Bax or caspases (Uren et al., 1996, Deveraux et al., 1998, Duckett et al., 1996, Liston et al., 1996), and is able to directly bind to caspases 3 and 7 and pro-caspase 9 (Deveraux et al., 1997, Deveraux et al., 1998). However, unlike the situation with DIAP1, there are no pro-apoptotic signalling molecules known that directly inhibit mammalian IAP function.

### BRIEF SUMMARY OF THE INVENTION

The present invention is predicated in part on the discovery of a pro-apoptotic polypeptide hereinafter referred to as DIABLO, which inhibits the activity of IAPs, including animal and viral IAPs. The inventors have reduced this discovery to practice in isolated molecules for use in screening for modulatory agents that modulate the level and/or functional activity of DIABLO, in methods for detecting cell death or apoptosis, in methods for diagnosis of conditions relating to the expression or activation of DIABLO, and in compositions for treating and/or preventing such DIABLO-related conditions, as described hereinafter.

Accordingly, in one aspect of the invention, there is provided an isolated polypeptide, or a biologically active fragment thereof, or a variant or derivative of these, and preferably a fragment at least 8 amino acids in length, said polypeptide comprising a member selected from the group consisting of:

(a) the sequence set forth in SEQ ID NO:2;

(b) the sequence set forth in SEQ ID NO:6;

(c) the sequence set forth in SEQ ID NO:7; and

(d) the sequence set forth in SEQ ID NO:8.

Preferably, the isolated polypeptide comprises the sequence set forth in SEQ ID NO:2 or 6.

In one embodiment, the isolated polypeptide comprises a leader peptide. In a preferred embodiment of this type, the leader peptide comprises residues 1 through 60 of the sequence set forth in SEQ ID NO:2, or residues 1 through 25 of the sequence set forth in SEQ ID NO:7. In an especially preferred embodiment of this type, the isolated polypeptide preferably comprises the sequence set forth in any one of SEQ ID NO:2 or 7.

In an alternate embodiment, the isolated polypeptide comprises a leader peptide and has a molecular mass of about 29-kDa. In a preferred embodiment of this type, the polypeptide has an isoelectric point (pI) of about 6.1.

In another embodiment, the polypeptide corresponds to a mature or N-terminally processed polypeptide. In a preferred embodiment of this type, the mature polypeptide comprises the sequence set forth in any one of SEQ ID NO:6 or 8. In another preferred embodiment, the mature polypeptide has a molecular mass of 23-kDa and a pI of 5.4.

In one embodiment, the biologically active fragment is selected from any one or more of residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232 and 228-237 of SEQ ID NO:2.

In another aspect, the invention contemplates an isolated polynucleotide encoding a polypeptide, fragment, variant or derivative as broadly described above. In a preferred embodiment, the polynucleotide comprises the sequence set forth in SEQ ID NO:3, or a biologically active fragment thereof, or a polynucleotide variant of these, and preferably, a fragment at least 24 nucleotides in length.

The polynucleotide preferably comprises a nucleotide sequence encoding a leader peptide. Suitably, said nucleotide sequence comprises nucleotides 1 through 180 of the sequence set forth in SEQ ID NO:3, or a polynucleotide variant of these. In such a case, the polynucleotide preferably comprises the sequence set forth in SEQ ID NO:1 or 3.

Preferably, the variant is obtained from an animal, including mammals and marine animals.

In another aspect, the invention contemplates a vector comprising a polynucleotide as broadly described above.

In yet another aspect, the invention features an expression vector comprising a polynucleotide as broadly described above wherein the polynucleotide is operably linked to a regulatory polynucleotide.

In a further aspect, the invention provides a host cell containing an expression vector as broadly described above.

The invention also contemplates a method of producing a recombinant polypeptide, fragment, variant or derivative as broadly described above, comprising:

culturing a host cell containing an expression vector as broadly described above such that said recombinant polypeptide, fragment, variant or derivative is expressed from said polynucleotide; and

isolating said recombinant polypeptide, fragment, variant or derivative.

In a further aspect, the invention encompasses a method of producing a biologically active fragment as broadly described above, comprising:

introducing a fragment of the polypeptide or a polynucleotide from which the fragment can be expressed into a cell; and

detecting cell death which is indicative of said fragment being a biologically active fragment.

In yet another aspect, the invention envisions a method of producing a biologically active fragment as broadly described above, comprising:

contacting an IAP with a fragment of the polypeptide; and

detecting a reduction in activity of the IAP which is indicative of said fragment being a biologically active fragment.

In yet another aspect, the invention contemplates a method of producing a polypeptide variant of a parent polypeptide comprising the sequence set forth in any one of SEQ ID NO:2, 6, 7 or 8, or biologically active fragment thereof, comprising:

providing a modified polypeptide whose sequence is distinguished from the parent polypeptide by substitution, deletion or addition of at least one amino acid;

contacting an IAP with the modified polypeptide; and

detecting a reduction in activity of the IAP which is indicative of the modified polypeptide being said polypeptide variant.

According to another aspect of the invention, there is provided a method of producing a polypeptide variant of a parent polypeptide comprising the sequence set forth in any one of SEQ ID NO:2, 6, 7 or 8, or biologically active fragment thereof, comprising:

introducing into a cell a modified polypeptide as described above or a polynucleotide from which said modified polypeptide can be expressed;

detecting cell death which is indicative of the modified polypeptide being a said polypeptide variant.

Undesirable activation or inactivation of apoptosis has been associated with many human conditions including diseases such as cancer, vascular disease, hepatic disease, autoimmune disease and neurodegenerative diseases. Accordingly, the isolated polypeptides and polynucleotides as broadly described above can be used to provide both drug targets and regulators to promote or inhibit apoptosis and to provide diagnostic markers for apoptosis during normal or disease stages, e.g. using detectable polypeptides and polynucleotides as broadly described above, or using detectable agents which interact specifically with those polypeptides or polynucleotides.

Thus, in another aspect, the invention extends to a method of screening for an agent which modulates cell death, said method comprising:

contacting a preparation comprising a polypeptide as broadly described above or a genetic sequence encoding said polypeptide with a test agent; and

detecting a change in the level and/or functional activity of said polypeptide or an expression product of said genetic sequence.

In another aspect, the invention resides in the use of a polypeptide, fragment, variant or derivative according to the present invention to produce an antigen-binding molecule that binds specifically to the said polypeptide, fragment, variant or derivative.

In yet another aspect, the invention provides antigen-binding molecules so produced.

According to another aspect of the invention, there is provided a method of detecting in a biological sample a polypeptide, fragment, variant or derivative as broadly described above, comprising:

contacting the sample with an antigen-binding molecule as broadly described above; and

detecting the presence of a complex comprising said antigen-binding molecule and said polypeptide, fragment, variant or derivative in said contacted sample.

In yet another aspect of the invention, there is provided a method of detecting in a biological sample a polypeptide, fragment, variant or derivative as broadly described above, comprising:

contacting the sample with an IAP which specifically binds to said polypeptide, fragment, variant or derivative; and

detecting the presence of a complex comprising said IAP and said polypeptide, fragment, variant or derivative in said contacted sample.

In another aspect, the invention envisions a method for detecting a specific polypeptide or polynucleotide sequence, comprising detecting a sequence of:

SEQ ID NO:2 or 6, or a fragment thereof at least 8 amino acids residues in length; or

SEQ ID NO:1 or 3, or a fragment thereof at least 24 nucleotides in length;

In yet another aspect, there is provided a method for detecting a polypeptide, fragment, variant or derivative as broadly described above, comprising:

detecting expression in a cell of a polynucleotide encoding said polypeptide, fragment, variant or derivative.

In yet another aspect of the invention, there is provided a method for modulating cell death, said method comprising contacting said cell with an agent for a time and under conditions sufficient to modulate the level and/or functional activity of a polypeptide as broadly described above.

In a preferred embodiment, the agent decreases the level and/or functional activity of said polypeptide.

In another aspect, the invention resides in a composition for treatment and/or prophylaxis of a condition associated with expression or activation of DIABLO, said composition comprising an agent which reduces the level and/or functional activity of a polypeptide as broadly described above, together with a pharmaceutically acceptable carrier.

According to another aspect of the invention, there is provided a method for treatment and/or prophylaxis of a condition associated with expression or activation of DIABLO, said method comprising administering to a patient in need of such treatment a therapeutically effective amount of an agent as broadly described above for a time and under conditions sufficient to modulate the level and/or functional activity of a polypeptide as broadly described above.

Suitably, the condition is selected from any one or more of cancer, vascular disease, hepatic disease, autoimmune disease and neurodegenerative disease. In a preferred embodiment of this type, the condition is tissue damage, including muscle tissue damage associated with heart attack and hepatic tissue damage associated with a liver disease.

The invention also encompasses the use of the polypeptide, fragment, variant or derivative as well as the modulatory agents as broadly described above in the study of cell death or apoptosis, and the treatment and/or prevention of conditions associated with cell death or apoptosis.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows detection of MIHA-interacting proteins. (A). Flag-MIHA (C-term) and Flag-DQMD (N-term) were immunoprecipitated from ³⁵S-labelled lysates of transiently transfected 293T cells using Flag specific mAb M2 coupled agarose beads. The immunoprecipitate was analysed by IPG/SDS PAGE and proteins visualised by autoradiography. Four proteins, #1 to #4, were detected which specifically immunoprecipitate with Flag-MIHA but not the control protein Flag-DQMD. Flag-tagged proteins are indicated by ‘F’. (B) Immunoprecipitates prepared from ³⁵S labelled lysates of control NT2 cells and an NT2 cell line stably expressing Flag-MIHA (N-term) were examined by IPG/SDS PAGE for MIHA. interacting proteins. Flag-MIHA migrated as a series of small spots and is indicated by the ‘F’. Co-immunoprecipitated protein spot #4 is clearly visible.

FIG. 2 shows identification of DIABLO. (A) Flag-MIHA was purified from transiently transfected 293T cells and with associated proteins was separated by 2D IPG/SDS PAGE and visualised with Coomassie blue. Protein #4 (DIABLO) was extracted from the gel for peptide analysis. (B) Capillary column RP-HPLC/ESI-MS/MS of a tryptic digest of DIABLO. The unfractionated peptide mixture (30 μL, ˜0.2 pmol from a total volume of ˜100 μL) was applied to the 0.2 mm ID column at 6 μ/min. The column was developed at 1.6 μ/min using a linear 60-min gradient from 0-100% B, where solvent A was 0.1% aqueous trifluoroacetic acid, and solvent B was 60% acetonitrile in aqueous 0.1% aqueous trifluoroacetic acid. Peptides were sequenced by collision-induced dissociation (CID) of their [M+H]⁺ and [M+2H]²⁺ ions. Peaks labelled with an asterisk were identified as trypsin autodigestion peptides. (C) Collision-induced dissociation—MS/MS spectrum of the singly-charged ion of tryptic peptide T1 (m/z 851.8). The amino acid sequence NH(L/I)(Q/K)(L/I)VK was determined by interpretation of the b- (italic text) and y-type (normal text) product ion series as shown (since the residues L/I are isobaric and K/Q have the same nominal mass, these amino acid residue pairs were not able to be differentiated using the instrumentation employed in this study). (D) Collision-induced dissociation—MS/MS spectrum of the doubly charged ion of tryptic: peptide T5 (m/z 1011.6). The amino acid sequence

XXSEEEDEVW(Q/K)V(L/I)(L/I)GAR was determined by manual de novo interpretation of the b- (italic text) and y-type (normal text) product ion series [Roepstorff and Fohlman, 1984] as shown. (X=unknown amino acid; since the residues L/I are isobaric and K/Q have the same nominal mass, these amino acid residue pairs were not able to be differentiated using the instrumentation employed in this study). Following identification of the cDNA, the unidentified residues XX were shown to correspond to MetAsn. The difference in observed (2021.6 D) versus calculated (2005.2 D) mass suggests that the methionine was present as the sulfoxide. Using the same approach, the following partial sequences were obtained for T2-T4 and T6-T7: T2 (obs. mass 1459.6 D . . . PHS(L/I) . . . A(L/I)M(so)R) note that M(so) corresponds to methionine sulfoxide; T3 (obs. mass 1339.3 D ((Q/K)(L/I))VEEVH(QIK)(L/I)SR) Note that the order of the two N-terminal amino acids was not able to be determined; T4, (obs. mass 1444.1 D . . . PHS(L/I)S . . . ); T6 (obs. mass 3376.4 D . . . AAEAAY(QIK)TGAD(Q/K)A . . . ); T7 (obs. mass 3153.9 D . . . (L/I)S(Q/K)TTYA(L/I)(L/I)EA(LII) . . . ). Inspection of the identified protein sequence determined the complete sequences of peptides T1-T7 as; T1, NHIQLVK, calc. mass 851.0; T2, SEPHSLSSEALM(so)R calc. mass 1459.6; T3, LQVEEVHQLSR calc. mass 1337.5; T4, SEPHSLSSEALMR calc. mass 1443.6; T5, M(so)NSEEEDEVWQVIIGAR calc. mass 2021.2; T6, LETTWM(so)TAVGLSEM(so)AAEAAYQTGADQASITAR calc mass 3377.3; T7, AVSLVTDSTSTFLSQTTYALIEAITEYTK calc. mass 3154.5. (E) Comparison of mouse and human DIABLO. The human sequences shown correspond to DNA sequences AA3038, T53449, T53449, AA3257 and AA1299. Human DIABLO peptide sequences identified by mass spectroscopic sequencing are underlined. Cleavage of the N terminal fragment must occur upstream of the first peptide, i.e. at residue 60 or before.

FIG. 3 shows expression of DIABLO in adult mouse tissues. Northern blots of polyA mRNA were hybridised with a probe encompassing the entire coding region of DIABLO. The blot was stripped and rehybridised with a β-actin probe.

FIG. 4 depicts specific interaction of MIHA with DIABLO. Anti-HA (A) and anti-Flag (3) immunoprecipitates prepared from ³⁵S labelled lysates of transiently transfected 293T cells were analysed by IPG/SDS PAGE. Flag-tagged proteins are denoted by ‘F’, HA-tagged proteins by ‘H’, endogenous DIABLO by E and untagged transfected DIABLO (unt.DIABLO) by ‘U’. An arrow indicates where the negative control protein Flag-DQMD would normally migrate. (C) Flag-MIHA (C-term) and Flag-DQMD were immunoprecipitated from transiently transfected 293T cells and interaction with co-transfected molecules HA-DIABLO and control HA-tagged proteins FLN29 and ZAP 1 examined by Western blot analysis with anti-HA antibody. Directly immunoprecipitated Flag-tagged proteins were subsequently demonstrated by re-probing the membrane with anti-Flag MAb and expression of the HA-tagged proteins confirmed by anti-HA Western blot analysis of whole cell lysate (WCL). (D) Immunoprecipitates of HA-DIABLO and control protein HA-FLN29 from transiently transfected 293T cells were examined for co-immunoprecipitating Flag-MIHA or Flag-DQMD by Western blot analysis with anti-Flag antibody as indicated. Flag-MIHA can be seen in the HA-DIABLO immunoprecipitate migrating just above the immunoglobulin (Ig) band. The directly immunoprecipitated HA tagged proteins were subsequently demonstrated by re-probing of the membrane with anti-HA antibody. Some residual Flag-MIHA signal is present in lane 1 from the previous blot. Expression of the Flag-tagged proteins was confirmed by anti-Flag Western blot analysis of WCL.

FIG. 5 shows interaction of DIABLO with mammalian IAPs MIHA, MIHB, MIHC and OpIAP. Flag-epitope tagged IAPs and control Flag-DQMD were immunoprecipitated from transiently transfected 293T cells and the immunoprecipitates examined for co-transfected HA-DIABLO by anti-HA Western blot analysis. The directly immunoprecipitated proteins were then demonstrated by sequential probing of the membrane with anti-Flag, anti-cIAP1 and anti-cIAP2 antibodies. The signals in lanes 1 and 6 of the anti-cIAP1 Western blot represent residual anti-Flag signals from Flag-MIHA and Flag-DQMD respectively. Expression of HA-DIABLO was confirmed by anti-HA Western of WCL.

FIG. 6 shows subcellular localisation of DIABLO and MIHA. 293T cells transiently transfected with Flag-MIHA and HA-DIABLO were lysed by passaging through a 27 G needle in a hypotonic buffer. The extract was then separated on a 50-10% sucrose gradient and collected fractions examined for Flag-MIHA, HA-DIABLO and a series of other proteins by Western blot analysis as indicated.

FIG. 7 shows MIHA can protect NT2 cells against UV radiation and this protection can be antagonised by DIABLO. (A) NT2 cells stably expressing pEFLacZ, but not those stably expressing Bcl-2 or MIHA, undergo apoptosis when exposed to UV radiation. Cells were exposed to different doses of UV radiation and their viability determined by exclusion of propidium iodide (PI) 20 hours after exposure. Mean viability ±2 SEM of 3 independent Bcl-2 clones, 3 independent MIHA clones and 4 independent pEFLacZ NT2 clones are shown. (B) NT2 cells stably expressing MIHA were transiently transfected with pEGFP and either pEFLacZ or pEFDIABLO expression constructs. After 48 hr the cells were exposed to 25 J/M2 UV radiation, and 8 hr later analysed by microscopy (B) and flow cytometry (C). NT2 cells stably expressing MIHA, Bcl-2 or pEFLac Z were transiently transfected with either pEFLacZ or pEFDIABLO together with a GFP encoding vector. Apoptosis of green (transfected) cells was determined by annexin V staining. Results represent the mean ±2 SEM of three independent experiments for 3 independent MIHA lines, 2 Bcl-2 lines and 1 pEFLacZ line.

### BRIEF DESCRIPTION OF THE SEQUENCES: SUMMARY TABLE

TABLE A

| SEQUENCE ID | | | |
|---|---|---|---|
| NUMBER | SEQUENCE | LENGTH | |
| SEQ ID NO: 1 | Full-length murine cDNA encoding | 1356 | bases |
| | DIABLO | | |
| SEQ ID NO: 2 | Full-length murine DIABLO poly- | 237 | residues |
| | peptide encoded by SEQ ID NO: 1 | | |
| SEQ ID NO: 3 | Coding sequence for full-length | 714 | bases |
| | murine DIABLO polypeptide | | |
| SEQ ID NO: 4 | Polypeptide encoded by | 237 | residues |
| | SEQ ID NO: 3 | | |
| SEQ ID NO: 5 | Polynucleotide encoding mature | 534 | bases |
| | murine DIABLO polypeptide | | |
| SEQ ID NO: 6 | Mature murine DIABLO polypeptide | 177 | residues |
| | encoded by SEQ ID NO: 5 | | |
| SEQ ID NO: 7 | Partial-length human DIABLO | 202 | residues |
| | polypeptide | | |
| SEQ ID NO: 8 | Full-length mature human DIABLO | 177 | residues |
| | polypeptide | | |
| SEQ ID NO: 9 | Partial rat DIABLO polypeptide | 84 | residues |
| SEQ ID NO: 10 | Partial flounder DIABLO polypeptide | 73 | residues |

### DETAILED DESCRIPTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to molecular masses or PIs that vary by as much as 30%, preferably by as much as 20%, and more preferably by as much as 10% to a reference molecular mass or pI.

By “agent” is meant a naturally occurring or synthetically produced molecule which interacts either directly or indirectly with a target member, the level and/or functional activity of which is to be modulated.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically” and the like refers to molecules, including antigen-binding molecules, that bind the polypeptide or polypeptide fragments of the invention but do not significantly bind to homologous prior art polypeptides.

By “biologically active fragment” is meant a fragment of a full-length parent polypeptide which fragment retains the activity of the parent polypeptide. A biologically active fragment will, therefore, inter alia promote cell death or apoptosis or elicit an immunogenic response to produce elements (e.g., antigen-binding molecules) that specifically bind to the parent polypeptide. As used herein, the term “biologically active fragment ” includes deletion mutants and small peptides, for example of at least 8, preferably at least 10, more preferably at least 15, even more preferably at least 20 and even more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines ” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. Preferably, the biological sample is a tissue biopsy, more preferably a muscle or liver tissue biopsy.

Throughout this specification, unless the context requires otherwise, the words “comprise ”, “comprises ” and “comprising” will be understood to imply the inclusion of a stated step or element or group of steps or elements but not the exclusion of any other step or element or group of steps or elements.

As used herein, the term “function” refers to a biological, enzymatic, or therapeutic function.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that will have apoptotic activity and promote cell death.

“Homology” refers to the percentage number of amino acids that are identical or constitute conservative substitutions as defined in Table A infra. Homology may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant that transcriptional and translational regulatory nucleic acids are positioned relative to a polypeptide-encoding polynucleotide in such a manner that the polynucleotide is transcribed and the polypeptide is translated.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. Accordingly, polypeptide variants as used herein encompass polypeptides that will promote cell death or apoptosis.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide ” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide ” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions ” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences. Other examples of stringency conditions are described in section 3.3.

By “therapeutically effective amount”, in the context of the treatment of a condition associated with expression DIABLO, is meant the administration of that amount of modulatory agent to an individual in need of such treatment, either in a single dose or as part of a series, that is effective for treatment of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

2. Isolated Polypeptides, Biologically Active Fragments, Polypeptide Variants and Derivatives

2.1. Isolated Polypeptides of the Invention

The invention features an isolated pro-apoptotic polypeptide, designated DIABLO, comprising the sequence set forth in any one of SEQ ID NO:2, 6, 7 or 8. SEQ ID NO:2 corresponds to a putative full-length murine polypeptide comprising a putative leader peptide (from residue 1 to about residue 60) and a mature or N-terminally processed polypeptide (from about residue 61 through 237). The unprocessed polypeptide has a molecular mass of about 29-kDa and a pI of about 6.1. The processed or mature polypeptide set forth in SEQ ID NO:6 has a molecular mass of about 23-kDa and a pI of about 5.4. SEQ ID NO:7 corresponds to a partial human polypeptide based on sequence alignment with the murine counterpart. This human polypeptide comprises a putative partial leader peptide (from residue 1 to about residue 25) and a full-length mature or N-terninally processed polypeptide (from about residue 26 through 202). SEQ ID NO:8 corresponds to the mature human polypeptide.

2.2. Biologically Active Fragments

Biologically active fragments may be produced according to any suitable procedure known in the art. For example, a suitable method may include first producing a fragment of said isolated polypeptide and then testing the fragment for the appropriate biological activity. In one embodiment, biological activity of the fragment may be tested by introducing a fragment of the polypeptide, or a polynucleotide from which the fragment can be expressed, into a cell and detecting cell death which is indicative of said fragment being a biologically active fragment. In a preferred embodiment, the cell is the human neuronal cell line NT2, which undergoes apoptosis when subjected to UV radiation (as described more fully hereinafter in Example 13, with particular reference to FIG. 7A). In this connection, NT2 cells stably or transiently transfected with vectors from which one or more IAPs are expressed do not undergo apoptosis when exposed to UV radiation. This assay, therefore, allows one to (a) test to IAP activity, and (b) by introduction of an additional expression construct, test for IAP inhibitory proteins, such as DIABLO (as described more fully hereinafter in Example 13, with particular reference to FIGS. 7B and 7C).

Alternatively, a suitable fragment may be tested for biological activity by contacting an IAP with a fragment of the polypeptide and detecting a reduction in activity of the IAP, which is indicative of said fragment being a biologically active fragment. Suitably, the IAP includes, but is not restricted to, a mammalian IAP homologue such as MIHA, MIHB, MIHC and the baculoviral IAP from Orygia pseudotsugata, OpIAP. Preferably, the IAP is MIHA.

The invention also extends to biological fragments of the above polypeptides, which can elicit an immune response in an animal and preferably in a heterologous animal from which the polypeptide is obtained. For example exemplary polypeptide fragments of 10 residues in length, which could elicit an immune response, include but are not limited to residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232 and 228-237 of SEQ ID NO:2.

2.3. Polypeptide Variants

The invention also contemplates polypeptide variants of the polypeptides of the invention wherein said variants promote apoptosis or cell death. Suitable methods of producing polypeptide variants include replacing at least one amino acid of a parent polypeptide comprising the sequence set forth in any one of SEQ ID NO:2, 6, 7 or 8, or a biologically active fragment thereof, with a different amino acid to produce a modified polypeptide, contacting an IAP with the modified polypeptide and detecting a reduction in activity of the IAP, which is indicative of the modified polypeptide being said polypeptide variant.

Alternatively, a polypeptide variant according to the invention may be identified by producing a modified polypeptide by replacing at least one amino acid of a parent polypeptide comprising the sequence set forth in any one of SEQ ID NO:2, 6, 7 or 8, or a biologically active fragment thereof, with a different amino acid. Alternatively, a polynucleotide is constructed from which this modified polypeptide can be expressed. The modified polypeptide or the polynucleotide is introduced into a cell and if cell death is detected, then this indicates that the modified polypeptide is a polypeptide variant.

In general, variants will be at least 80% homologous, more suitably at least 85%, preferably at least 90%, and more preferably at least 95% homologous to a polypeptide as for example shown in any one of SEQ ID NO:2, 6, 7 or 8 or in fragments thereof. For example, the inventors have identified two putative partial polypeptide variants from rat and flounder, the sequences of which are set forth in SEQ ID NO:9 and 10, respectively.

2.4. Methods of Producing Polypeptide Variants

2.4.1. Mutagenesis

Polypeptide variants according to the invention can be identified either rationally, or via established methods of mutagenesis (see, for example, Watson, J. D. et al., “MOLECULAR BIOLOGY OF THE GENE”, Fourth Edition, Benjamin/Cummings, Menlo Park, Calif., 1987). Significantly, a random mutagenesis approach requires no a priori information about the gene sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow, R. 1986, J Prot. Eng. 1: 7-16; Knowles, J. R., 1987, Science 236: 1252-1258; Shaw, W. V., 1987, Biochem. J. 246: 1-17; Gerit, J. A. 1987, Chem. Rev. 87: 1079-1105).

Alternatively, where a particular sequence alteration is desired, methods of site-directed mutagenesis can be employed. Thus, such methods may be used to selectively alter only those amino acids of the protein that are believed to be important (Craik, C. S., 1985, Science 228: 291-297; Cronin, et al., 1988, Biochem. 27: 4572-4579; Wilks, et al., 1988, Science 242: 1541-1544).

Variant peptides or polypeptides, resulting from rational or established methods of mutagenesis or from combinatorial chemistries as hereinafter described, may comprise conservative amino acid substitutions. Exemplary conservative substitutions in a polypeptide or polypeptide fragment according to the invention may be made according to the following table:

TABLE A

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE A. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (eg., Gly).

What constitutes suitable variants may be determined by conventional techniques. For example, nucleic acids encoding a polypeptide according to any one of SEQ ID NO:2, 6, 7 or 8 can be mutated using either random mutagenesis for example using transposon mutagenesis, or site-directed mutagenesis as described, for example, in Section 3.2 infra.

2.4.2. Peptide Libraries Produced by Combinatorial Chemistry

A number of facile combinatorial technologies can be utilised to synthesise molecular libraries of immense diversity. In the present case, variants of a polypeptide, or preferably a polypeptide fragment according to the invention, can be synthesised using such technologies. Variants can be screened subsequently using the methods described in Section 2.2.

Preferably, soluble synthetic peptide combinatorial libraries (SPCLs) are produced which offer the advantage of working with free peptides in solution, thus permitting adjustment of peptide concentration to accommodate a particular assay system. SPCLs are suitably prepared as hexamers. In this regard, a majority of binding sites is known to involve four to six residues. Cysteine is preferably excluded from the mixture positions to avoid the formation of disulfides and more difficult-to-define polymers. Exemplary methods of producing SPCLs are disclosed by Houghten et al. (1991, Nature 354: 84-86; 1992, BioTechniques 13: 412-421), Appel et al. (1992, Immunomethods 1: 17-23), and Pinilla et al. (1992, BioTechniques 13: 901-905; 1993, Gene 128: 71-76).

Preparation of combinatorial synthetic peptide libraries may employ either t-butyloxycarbonyl (t-Boc) or 9-fluorenylmethyloxycarbonyl (Fmoc) chemistries (see Chapter 9.1, of Coligan et al., supra; Stewart and Young, 1984, Solid Phase Peptide Synthesis, 2nd ed. Pierce Chemical Co., Rockford, Ill; and Atherton and Sheppard, 1989, Solid Phase Peptide Synthesis: A Practical Approach. IRL Press, Oxford) preferably, but not exclusively, using one of two different approaches. The first of these approaches, suitably termed the “split-process-recombine” or “split synthesis” method, was described first by Furka et al. (1988, 14th Int. Congr. Biochem., Prague, Czechoslovakia 5: 47; 1991, Int. J Pept. Protein Res. 37: 487-493) and Lam et al. (1991, Nature 354: 82-84), and reviewed later by Eichler et al. (1995, Medicinal Research Reviews 15(6): 481-496) and Balkenhohl et al. (1996, Angew. Chem. Int. Ed. Engl. 35: 2288-2337). Briefly, the split synthesis method involves dividing a plurality of solid supports such as polymer beads into n equal fractions representative of the number of available amino acids for each step of the synthesis (e.g., 20 L-amino acids), coupling a single respective amino acid to each polymer bead of a corresponding fraction, and then thoroughly mixing the polymer beads of all the fractions together. This process is repeated for a total of x cycles to produce a stochastic collection of up to N^{x} different compounds. The peptide library so produced may be screened for example with a suitably labelled antigen-binding molecule that binds specifically to a polypeptide according to any one of SEQ ID NO:2, 6, 7 or 8. Upon detection, some of the positive beads are selected for sequencing to identify the active peptide. Such peptide may be subsequently cleaved from the beads, and assayed using the same antigen-binding molecule to identify the most active peptide sequence.

The second approach, the chemical ratio method, prepares mixed peptide resins using a specific ratio of amino acids empirically defined to give equimolar incorporation of each amino acid at each coupling step. Each resin bead contains a mixture of peptides. Approximate equimolar representation can be confirmed by amino acid analysis (Dooley and Houghten, 1993, Proc. Natl. Acad. Sci. U.S.A. 90: 10811-10815; Eichler and Houghten, 1993, Biochemistry 32: 11035-11041). Preferably, the synthetic peptide library is produced on polyethylene rods, or pins, as a solid support, as for example disclosed by Geysen et al. (1986, Mol. Immunol. 23: 709-715). An exemplary peptide library of this type may include octapeptides in which the third and fourth positions represent defined amino acids selected from natural and unnatural amino acids, and in which the remaining six positions represent a randomized mixture of amino acids. This peptide library can be represented by the formula Ac-X₁X₂O₁O₂X₃X₄X₅X₆-Sₛ, where O_{1 and O}₂ are each defined amino acids, X₁, through X₆ are a randomized mixture of the same or different amino acids and Sₛ is the solid support. Peptide mixtures remain on the pins when assayed against a soluble receptor molecule. For example, the peptide library of Geysen (1986, Immun. Today 6: 364-369; and Geysen et al., Ibid), comprising for example dipeptides, is first screened for the ability to bind to a target molecule. The most active dipeptides are then selected for an additional round of testing comprising linking, to the starting dipeptide, an additional residue (or by internally modifying the components of the original starting dipeptide) and then screening this set of candidates for the desired activity. This process is reiterated until the binding partner having the desired properties has been determined.

2.4.3. Alanine Scanning Mutagenesis

In one embodiment, the invention herein utilises a systematic analysis of a polypeptide or polypeptide fragment according to the invention to determine the residues in the polypeptide or fragment that are involved in the interaction with an IAP. Such analysis is conveniently performed using recombinant DNA technology. In general, a DNA sequence encoding the polypeptide or fragment is cloned and manipulated so that it may be expressed in a convenient host. DNA encoding the polypeptide or fragment can be obtained from a genomic library, from cDNA derived from mRNA in cells expressing the said polypeptide or fragment, or by synthetically constructing the DNA sequence (Sambrook et al., supra; Ausubel et al., supra).

The wild-type DNA encoding the polypeptide or fragment is then inserted into an appropriate plasmid or vector as described herein. In particular, prokaryotes are preferred for cloning and expressing DNA sequences to produce variants of the polypeptide or fragment. For example, E. coli K12 strain 294 (ATCC No. 31446) may be used, as well as E. coli B, E. coli X1776 (ATCC No. 31537), and E. coli c600 and c600hfl, and E. coli W3110 (F⁻, γ⁻, prototrophic, ATCC No. 27325), bacilli such as Bacillus subtilis, and other enterobacteriaceae such as Salmonella typhimurium or Serratia marcescens, and various Pseudomonas species. A preferred prokaryote is E. coli W3110 (ATCC 27325).

Once the polypeptide or fragment is cloned, site-specific mutagenesis as for example described by Carter et al. (1986, Nucl. Acids. Res., 13: 4331) or by Zoller et al. (1987, Nucl. Acids Res., 10: 6487), cassette mutagenesis as for example described by Wells et al. (1985, Gene, 34: 315), restriction selection mutagenesis as for example described by Wells et al. (1986, Philos. Trans. R. Soc. London SerA, 317: 415), or other known techniques may be performed on the cloned DNA to produce the variant DNA that encodes for the changes in amino acid sequence defined by the residues being substituted. When operably linked to an appropriate expression vector, variants are obtained. In some cases, recovery of the variant may be facilitated by expressing and secreting such molecules from the expression host by use of an appropriate signal sequence operably linked to the DNA sequence encoding the variant. Such methods are well known to those skilled in the art. Of course, other methods may be employed to produce such polypeptides or fragments such as the in vitro chemical synthesis of the desired polypeptide variant (Barany et al. In The Peptides, eds. E. Gross and J. Meienhofer (Academic Press: N.Y. 1979), Vol. 2, pp. 3-254).

Once the different variants are produced, they are contacted with an IAP and the interaction, if any, between this IAP and each variant is determined. These activities are compared to the activity of the parent polypeptide or fragment with the same IAP molecule to determine which of the amino acid residues in the active domain are involved in the interaction with the IAP. The scanning amino acid used in such an analysis may be any different amino acid from that substituted, i.e., any of the 19 other naturally occurring amino acids.

The interaction between the IAP, and parent and variant, respectively, can be measured by any convenient assay as for example described herein. While any number of analytical measurements may be used to compare activities, a convenient one for binding of IAP is the dissociation constant K_{d} of the complex formed between the variant and IAP as compared to the K_{d} for the parent polypeptide or fragment. Generally, a two-fold increase or decrease in K_{d} per analogous residue substituted by the substitution indicates that the substituted residue(s) is active in the interaction of the parent polypeptide or fragment with the target IAP.

When a suspected or known active amino acid residue is subjected to scanning amino acid analysis, the amino acid residues immediately adjacent thereto should be scanned. Three residue-substituted polypeptides can be made. One contains a scanning amino acid, preferably alanine, at position N that is the suspected or known active amino acid. The two others contain the scanning amino acid at position N+1 and N−1. If each substituted polypeptide or fragment causes a greater than about two-fold effect on K_{d} for the receptor, the scanning amino acid is substituted at position N+2 and N−2. This is repeated until at least one, and preferably four, residues are identified in each direction which have less than about a two-fold effect on K_{d} or either of the ends of the parent polypeptide or fragment are reached. In this manner, one or more amino acids along a continuous amino acid sequence that are involved in the interaction with the particular IAP molecule can be identified.

The active amino acid residue identified by amino acid scan is typically one that contacts the IAP directly. However, active amino acids may also indirectly contact the IAP through salt bridges formed with other residues or small molecules such as H₂O or ionic species such as Na⁺, Ca⁺², Mg⁺², or Zn⁺².

In some cases, the substitution of a scanning amino acid at one or more residues results in a residue-substituted polypeptide which is not expressed at levels that allow for the isolation of quantities sufficient to carry out analysis of its activity with the IAP. In such cases, a different scanning amino acid, preferably an isosteric amino acid, can be used.

Among the preferred scanning amino acids are relatively small, neutral amino acids. Such amino acids include alanine, glycine, serine, and cysteine. Alanine is the preferred scanning amino acid among this group because it eliminates the side-chain beyond the beta-carbon and is less likely to alter the main-chain conformation of the variant. Alanine is also preferred because it is the most common amino acid. Further, it is frequently found in both buried and exposed positions (Creighton, The Proteins, W. H. Freeman & Co., N.Y.; Chothia, 1976, J. Mol. Biol., 150: 1). If alanine substitution does not yield adequate amounts of variant, an isosteric amino acid can be used. Alternatively, the following amino acids in decreasing order of preference may be used: Ser, Asn, and Leu.

Once the active amino acid residues are identified, isosteric amino acids may be substituted. Such isosteric substitutions need not occur in all instances and may be performed before any active amino acid is identified. Such isosteric amino acid substitution is performed to minimise the potential disruptive effects on conformation that some substitutions can cause. Isosteric amino acids are shown in the table below:

TABLE B

| Polypeptide Amino Acid | Isosteric Scanning Amino Acid |
|---|---|
| Ala (A) | Ser, Gly |
| Glu (E) | Gln, Asp |
| Gln (Q) | Asn, Glu |
| Asp (D) | Asn, Glu |
| Asn (N) | Ala, Asp |
| Leu (L) | Met, Ile |
| Gly (G) | Pro, Ala |
| Lys (K) | Met, Arg |
| Ser (S) | Thr, Ala |
| Val (V) | Ile, Thr |
| Arg (R) | Lys, Met, Asn |
| Thr (T) | Ser, Val |
| Pro (P) | Gly |
| Ile (I) | Met, Leu, Val |
| Met (M) | Ile, Leu |
| Phe (F) | Tyr |
| Tyr (Y) | Phe |
| Cys (C) | Ser, Ala |
| Trp (W) | Phe |
| His (H) | Asn, Gln |

The method herein can be used to detect active amino acid residues within different domains of a polypeptide or fragment according to the invention. Once this identification is made various modifications to the parent polypeptide or fragment may be made to modify the interaction between the parent polypeptide or fragment and an IAP.

2.4.4. Polypeptide or Peptide Libraries Produced by Phage Display

The identification of variants can also be facilitated through the use of a phage (or phagemid) display protein ligand screening system as for example described by Lowman, et al. (1991, Biochem. 30: 10832-10838), Markland, et al. (1991, Gene 109: 13-19), Roberts, et al. (1992, Proc. Natl. Acad. Sci. (U.S.A.) 89: 2429-2433), Smith, G. P. (1985, Science 228: 1315-1317), Smith, et al. (1990, Science 248: 1126-1128) and Lardner et al. (U.S. Pat. No. 5,223,409). In general, this method involves expressing a fusion protein in which the desired protein ligand is fused to the N-terminus of a viral coat protein (such as the M13 Gene III coat protein, or a lambda coat protein).

In one embodiment, a library of phage is engineered to display novel peptides within the phage coat protein sequences. Novel peptide sequences are generated by random mutagenesis of gene fragments encoding a polypeptide of the invention or biologically active fragment using error-prone PCR, or by in vivo mutation by E. coli mutator cells. The novel peptides displayed on the surface of the phage are placed in contact, with an IAP molecule. Phage that display coat protein having peptides that are capable of binding to an IAP are immobilised by such treatment, whereas all other phage can be washed away. After the removal of unbound phage, the bound phage can be amplified, and the DNA encoding their coat proteins can be sequenced. In this manner, the amino acid sequence of the embedded peptide or polypeptide can be deduced.

In more detail, the method involves (a) constructing a replicable expression vector comprising a first gene encoding a polypeptide or fragment of the invention, a second gene encoding at least a portion of a natural or wild-type phage coat protein wherein the first and second genes are heterologous, and a transcription regulatory element operably linked to the first and second genes, thereby forming a gene fusion encoding a fusion protein; (b) mutating the vector at one or more selected positions within the first gene thereby forming a family of related plasmids; (c) transforming suitable host cells with the plasmids; (d) infecting the transformed host cells with a helper phage having a gene encoding the phage coat protein; (e) culturing the transformed infected host cells under conditions suitable for forming recombinant phagemid particles containing at least a portion of the plasmid and capable of transforming the host, the conditions adjusted so that no more than a minor amount of phagemid particles display more than one copy of the fusion protein on the surface of the particle; (f) contacting the phagemid particles with an IAP molecule that binds to the parent polypeptide or fragment so that at least a portion of the phagemid particles bind to the IAP; and (g) separating the phagemid particles that bind from those that do not. Preferably, the method further comprises transforming suitable host cells with recombinant phagemid particles that bind to the IAP molecule and repeating steps (d) through (g) one or more times.

Preferably in this method the plasmid is under tight control of the transcription regulatory element, and the culturing conditions are adjusted so that the amount or number of phagemid particles displaying more than one copy of the fusion protein on the surface of the particle is less than about 1%. Also, preferably, the amount of phagemid particles displaying more than one copy of the fusion protein is less than 10% of the amount of phagemid particles displaying a single copy of the fusion protein. Most preferably, the amount is less than 20%.

Typically in this method, the expression vector will further contain a secretory signal sequence fused to the DNA encoding each subunit of the polypeptide and the transcription regulatory element will be a promoter system. Preferred promoter systems are selected from lac Z, λ_{PL}, tac, T7 polymerase, tryptophan, and alkaline phosphatase promoters and combinations thereof. Also, normally the method will employ a helper phage selected from M13K07, M13R408, M13-VCS, and Phi X 174. The preferred helper phage is M13K07, and the preferred coat protein is the M13 Phage gene III coat protein. The preferred host is E. coli, and protease-deficient strains of E. coli.

Repeated cycles of variant selection are used to select for higher and higher affinity binding by the phagemid selection of multiple amino acid changes that are selected by multiple selection cycles. Following a first round of phagemid selection, involving a first region or selection of amino acids in the ligand polypeptide, additional rounds of phagemid selection in other regions or amino acids of the ligand polypeptide are conducted. The cycles of phagemid selection are repeated until the desired affinity properties of the ligand polypeptide are achieved.

It will be appreciated that the amino acid residues that form the binding domain of the polypeptide or fragment may not be sequentially linked and may reside on different subunits of the polypeptide or fragment. That is, the binding domain tracks with the particular secondary structure at the binding site and not the primary structure. Thus, generally, mutations will be introduced into codons encoding amino acids within a particular secondary structure at sites directed away from the interior of the polypeptide so that they will have the potential to interact with the IAP.

The phagemid-display method herein contemplates fusing a polynucleotide encoding the polypeptide or fragment (polynucleotide 1) to a second polynucleotide (polynucleotide 2) such that a fusion protein is generated during transcription. Polynucleotide 2 is typically a coat protein gene of a phage, and preferably it is the phage M13 gene III coat protein, or a fragment thereof. Fusion of polynucleotides 1 and 2 may be accomplished by inserting polynucleotide 2 into a particular site on a plasmid that contains polynucleotide 1, or by inserting polynucleotide 1 into a particular site on a plasmid that contains polynucleotide 2.

Between polynucleotide 1 and polynucleotide 2, DNA encoding a termination codon may be inserted, such termination codons being UAG (amber), UAA (ocher), and UGA (opel) (see for example, Davis et al., Microbiology (Harper and Row: New York, 1980), pages 237, 245-247, and 274). The termination codon expressed in a wild-type host cell results in the synthesis of the polynucleotide 1 protein product without the polynucleotide 2 protein attached. However, growth in a suppressor host cell results in the synthesis of detectable quantities of fused protein. Such suppressor host cells contain a tRNA modified to insert an amino acid in the termination codon position of the mRNA, thereby resulting in production of detectable amounts of the fusion protein. Suppressor host cells of this type are well known and described, such as E. coli suppressor strain (Bullock et al., 1987, BioTechniques, 5: 376-379). Any acceptable method may be used to place such a termination codon into the mRNA encoding the fusion polypeptide.

The suppressible codon may be inserted between the polynucleotide encoding the polypeptide or fragment and a second polynucleotide encoding at least a portion of a phage coat protein. Alternatively, the suppressible termination codon may be inserted adjacent to the fusion site by replacing the last amino acid triplet in the polypeptide/fragment or the first amino acid in the phage coat protein. When the phagemid containing the suppressible codon is grown in a suppressor host cell, it results in the detectable production of a fusion polypeptide containing the polypeptide or fragment and the coat protein. When the phagemid is grown in a non-suppressor host cell the polypeptide or fragment is synthesised substantially without fusion to the phage coat protein due to termination at the inserted suppressible triplet encoding UAG, UAA, or UGA. In the non-suppressor cell the polypeptide is synthesised and secreted from the host cell due to the absence of the fused phage coat protein which otherwise anchored it to the host cell.

The polypeptide or fragment may be altered at one or more selected codons. An alteration is defined as a substitution, deletion, or insertion of one or more codons in the gene encoding the polypeptide or fragment that results in a change in the amino acid sequence as compared with the unaltered or native sequence of the said polypeptide or fragment. Preferably, the alterations will be by substitution of at least one amino acid with any other amino acid in one or more regions of the molecule. The alterations may be produced by a variety of methods known in the art. These methods include, but are not limited to, oligonucleotide-mediated mutagenesis and cassette mutagenesis as described for example in Section 3.2 infra.

For preparing the IAP molecule and binding it with the phagemid, the IAP molecule is attached to a suitable matrix such as agarose beads, acrylamide beads, glass beads, cellulose, various acrylic copolymers, hydroxyalkyl methacrylate gels, polyacrylic acid, polymethacrylic copolymers, nylon, neutral and ionic carriers, and the like. Attachment of the IAP molecule to the matrix may be accomplished by methods described in Methods Enzymol., 44: (1976), or by other means known in the art.

After attachment of the IAP molecule to the matrix, the immobilised IAP is contacted with the library of phagemid particles under conditions suitable for binding of at least a portion of the phagemid particles with the immobilised IAP or target. Normally, the conditions, including pH, ionic strength, temperature, and the like will mimic physiological conditions.

Bound phagemid particles (“binders”) having high affinity for the immobilised target are separated from those having a low affinity (and thus do not bind to the target) by washing. Binders may be dissociated from the immobilised target by a variety of methods. These methods include competitive dissociation using the wild-type ligand, altering pH and/or ionic strength, and methods known in the art.

Suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected.

2.4.5. Rational Drug Design

Variants of an isolated polypeptide according to the invention or a biologically active fragment thereof may also be obtained using the principles of conventional or of rational drug design as for example described by Andrews, et al (In: “PROCEEDINGS OF THE ALFRED BENZON SYMPOSIUM”, volume 28, pp. 145-165, Munksgaard, Copenhagen, 1990), McPherson, A. (1990, Eur. J. Biochem. 189: 1-24), Hol,. et al. (In: “MOLECULAR RECOGNITION: CHEMICAL AND BIOCHEMICAL PROBLEMS”, Roberts, S. M. (ed.); Royal Society of Chemistry; pp. 84-93, 1989), Hol, W. G. J. (1989, Arzneim-Forsch. 39: 1016-1018), Hol, W. G. J. (1986, Agnew Chem. Int. Ed. Engl. 25: 767-778).

In accordance with the methods of conventional drug design, the desired variant molecules are obtained by randomly testing molecules whose structures have an attribute in common with the structure of a parent polypeptide or biologically active fragment according to the invention. The quantitative contribution that results from a change in a particular group of a binding molecule can be determined by measuring the capacity of competition or cooperativity between the parent polypeptide or polypeptide fragment and the candidate polypeptide variant.

In one embodiment of rational drug design, the polypeptide variant is designed to share an attribute of the most stable three-dimensional conformation of a polypeptide or polypeptide fragment according to the invention. Thus, the variant may be designed to possess chemical groups that are oriented in a way sufficient to cause ionic, hydrophobic, or van der Waals interactions that are similar to those exhibited by the immuno-interactive polypeptide or polypeptide fragment. In a second method of rational design, the capacity of a particular polypeptide or polypeptide fragment to undergo conformational “breathing” is exploited. Such “breathing”—the transient and reversible assumption of a different molecular conformation—is a well-appreciated phenomenon, and results from temperature, thermodynamic factors, and from the catalytic activity of the molecule. Knowledge of the 3-dimensional structure of the polypeptide or polypeptide fragment facilitates such an evaluation. An evaluation of the natural conformational changes of a polypeptide or polypeptide fragment facilitates the recognition of potential hinge sites, potential sites at which hydrogen bonding, ionic bonds or van der Waals bonds might form or might be eliminated due to the breathing of the molecule, etc. Such recognition permits the identification of the additional conformations that the polypeptide or polypeptide fragment could assume, and enables the rational design and production of mimetic polypeptide variants that share such conformations.

The preferred method for performing rational mimetic design employs a computer system capable of forming a representation of the three-dimensional structure of the polypeptide or polypeptide fragment (such as those obtained using RIBBON (Priestle, J., 1988, J. Mol. Graphics 21: 572), QUANTA (Polygen), InSite (Biosyn), or Nanovision (American Chemical Society)). Such analyses are exemplified by Hol, et al. (In: “MOLECULAR RECOGNITION: CHEMICAL AND BIOCHEMICAL PROBLEMS”, supra, Hol, W. G. J. (1989, supra) and Hol, W. G. J., (1986, supra).

In lieu of such direct comparative evaluations of candidate polypeptide variants, screening assays may be used to identify such molecules. Such assays will preferably exploit the capacity of the variant to bind to an IAP and inhibit its activity.

2.5. Polypeptide Derivatives

With reference to suitable derivatives of the invention, such derivatives include amino acid deletions and/or additions to a polypeptide, fragment or variant of the invention, wherein said derivatives inhibit the activity of an IAP. “Additions ” of amino acids may include fusion of the polypeptides, fragments and polypeptide variants of the invention with other polypeptides or proteins. For example, it will be appreciated that said polypeptides, fragments or variants may be incorporated into larger polypeptides, and that such larger polypeptides may also be expected to inhibit IAP activity.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivatisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE C.

TABLE C

| Unnatural | Unnatural |
|---|---|
| α-aminobutyric acid | L-N-methylalanine |
| α-amino-α-methylbutyrate | L-N-methylarginine |
| Aminocyclopropane-carboxylate | L-N-methylasparagine |
| Aminoisobutyric acid | L-N-methylaspartic acid |
| Aminonorbornyl-carboxylate | L-N-methylcysteine |
| Cyclohexylalanine | L-N-methylglutamine |
| Cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilise 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C_{α} and C_{β} atoms of amino acids, by incorporation of C_{α} and N_{α}-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993, Biorganic & Medicinal Chemistry Letters 3: 437-44) who describes peptide cyclisation on TFA resin using trimethylsilyl (TMSE) ester as an orthogonal protecting group; Pallin and Tam (1995, J. Chem. Soc. Chem. Comm. 2021-2022) who describe the cyclisation of unprotected peptides in aqueous solution by oxime formation; Algin et al (1994, Tetrahedron Letters 35: 9633-9636) who disclose solid-phase synthesis of head-to-tail cyclic peptides via lysine side-chain anchoring; Kates et al (1993, Tetrahedron Letters 34: 1549-1552) who describe the production of head-to-tail cyclic peptides by three-dimensional solid phase strategy; Tumelty et al (1994, J. Chem. Soc. Chem. Comm. 1067-1068) who describe the synthesis of cyclic peptides from an immobilised activated intermediate, wherein activation of the immobilised peptide is carried out with N-protecting group intact and subsequent removal leading to cyclisation; McMurray et al (1994, Peptide Research 7: 195-206) who disclose head-to-tail cyclisation of peptides attached to insoluble supports by means of the side chains of aspartic and glutamic acid; Hruby et al (1994, Reactive Polymers 22: 231-241) who teach an alternate method for cyclising peptides via solid supports; and Schmidt and Langer (1997, J. Peptide Res. 49: 67-73) who disclose a method for synthesising cyclotetrapeptides and cyclopentapeptides. The foregoing methods may be used to produce conformationally constrained polypeptides that promote cell death or apoptosis or that bind to, or inhibit the activity of, an IAP.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

2.6. Methods of Preparing the Polypeptides of the Invention

Polypeptides of the inventions may be prepared by any suitable procedure known to those of skill in the art. For example, the polypeptides may be prepared by a procedure including the steps of:

(a) preparing a recombinant polynucleotide comprising a nucleotide sequence encoding a polypeptide comprising the sequence set forth in any one of SEQ ID NO:2, 6, 7 or 8, or variant or derivative of these, which nucleotide sequence is operably linked to transcriptional and translational regulatory nucleic acid;

(b) introducing the recombinant polynucleotide into a suitable host cell;

(c) culturing the host cell to express recombinant polypeptide from said recombinant polynucleotide; and

(d) isolating the recombinant polypeptide.

Suitably, said nucleotide sequence comprises the sequence set forth in any one of SEQ ID NO:1,3 or 5.

The recombinant polynucleotide preferably comprises either an expression vector that may be a self-replicating extra-chromosomal vector such as a plasmid, or a vector that integrates into a host genome.

The transcriptional and translational regulatory nucleic acid will generally be appropriate for the host cell used for expression. Numerous types of appropriate expression vectors and suitable regulatory sequences are known in the art for a variety of host cells.

Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide.

In order to express said fusion polypeptide, it is necessary to ligate a polynucleotide according to the invention into the expression vector so that the translational reading frames of the fusion partner and the polynucleotide coincide.

Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application.

Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the polypeptide, fragments, variants or derivatives of the invention may be synthesised using solution synthesis or solid phase synthesis as described, for example, in Chapter 9 of Atherton and Shephard (supra) and in Roberge et al (1995, Science 269: 202).

3. Polynucleotides of the Invention

3.1. Polynucleotides Encoding Polypeptides of the Invention

The invention further provides a polynucleotide that encodes a polypeptide, fragment, variant or derivative as defined above. Suitably, the polynucleotide comprises the entire sequence of nucleotides set forth in SEQ ID NO:1. SEQ ID NO:1 corresponds to a murine 1356 bp cDNA sequence (IMAGE consortium EST clone) deposited under GenBank Accession No AA276162. This sequence defines: (1) a 5′ untranslated region from nucleotide 1 through nucleotide 10 of SEQ ID NO:1; (2) an open reading frame from nucleotide 11 through nucleotide 724; and (3) a 3′ untranslated region from nucleotide 725 through nucleotide 1356. The aforementioned open reading frame encodes a precursor polypeptide comprising a putative leader peptide encoded by nucleotides 11 through 190, and a mature polypeptide encoded by nucleotides 191 through 724. Suitably, the polynucleotide comprises the sequence set forth in SEQ ID NO:3. SEQ ID NO:3 defines the aforementioned open reading frame and thus encodes the said precursor polypeptide. Preferably, the polynucleotide comprises the sequence set forth in SEQ ID NO:5, which corresponds to nucleotide 191 through nucleotide 1356 and thus encodes the said mature polypeptide. 3.2. Polynucleotides Variants

In general, polynucleotide variants according to the invention comprise regions that show at least 70%, more suitably at least 80%, preferably at least 90%, and most preferably at least 95% sequence identity over a reference polynucleotide sequence of identical size (“comparison window”) or when compared to an aligned sequence in which the alignment is performed by a computer homology program known in the art. What constitutes suitable variants may be determined by conventional techniques. For example, a polynucleotide according to any one of SEQ ID NO:1, 3 or 5 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983, DNA 2:183). Briefly, a polynucleotide according to any one of SEQ ID NO:1, 3 or 5 is altered by hybridising an oligonucleotide encoding the desired mutation to a template DNA, wherein the template is the single-stranded form of a plasmid or bacteriophage containing the unaltered or parent DNA sequence. After hybridisation, a DNA polymerase is used to synthesise an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridise properly to the single-stranded DNA template molecule.

The DNA template can be generated by those vectors that are either derived from bacteriophage M13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase I, is then added to synthesise the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al., supra, in particular Chapters 8.2A and 8.5.

Alternatively, suitable polynucleotide sequence variants of the invention may be prepared according to the following procedure:

(a) creating primers which are optionally degenerate wherein each comprises a portion of a reference polynucleotide encoding a reference polypeptide or fragment of the invention, preferably encoding the sequence set forth in any one of SEQ ID NO:2, 6, 7 or 8;

(b) obtaining a nucleic acid extract from an organism, which is preferably an animal, and more preferably a mammal, including a human; and

(c) using said primers to amplify, via nucleic acid amplification techniques, at least one amplification product from said nucleic acid extract, wherein said amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (“Molecular Cloning. A Laboratory Manual”, Cold Spring Harbour Press, 1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled addressee will recognise that a number of factors influence hybridisation. The specific activity of radioactively labelled polynucleotide sequence should typically be greater than or equal to about 108 dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention will hybridise to a reference polynucleotide under at least low stringency conditions. Reference herein to low stringency conditions includes and encompasses from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6 (log₁₀ M)+0.41 (%G+C)−0.63 (% formamide)−(600/length)

wherein: M is the concentration of Na⁺, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5×Denhardt's solution (0.1% ficoll, 0.1% polyvinylpyrollidone and 0.1% bovine serum albumin), 0.1% SDS and 200 mg/ml denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.).

Methods for detecting a labelled polynucleotide hybridised to an immobilised polynucleotide are well known to practitioners in the art. Such methods include autoradiography, phosphorimaging, and chemiluminescent, fluorescent and colorimetric detection.

4. Antigen-binding Molecules

The invention also contemplates antigen-binding molecules that bind specifically to the aforementioned polypeptides, fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Kohler and Milstein (1975, Nature 256, 495-497), or by more recent modifications thereof as described, for example, in Coligan et al., (1991, supra) by immortalising spleen or other antibody producing cells derived from a production species which has been inoculated with one or more of the polypeptides, fragments, variants or derivatives of the invention.

The invention also contemplates as antigen-binding molecules Fv, Fab, Fab′ and F(ab′)₂ immunoglobulin fragments.

Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Krebber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plückthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the filly folded Fv molecule the two residues will form a disulphide bond therebetween. Suitable methods of producing dsFv are described for example in (Glockscuther et al. Biochem. 29: 1363-1367; Reiter et al. 1994, J. Biol. Chem. 269:18327-18331; Reiter et al. 1994, Biochem. 33: 5451-5459; Reiter et al. 1994. Cancer Res. 54: 2714-2718; Webber et al. 1995, Mol Immunol. 32: 249-258).

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (i.e., having more than one antigen binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plinckthun, 1992, Biochem. 31: 1579-1584), or by use of domains (such as the leucine zippers jun and fos) that preferentially heterodimerise (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra).

The antigen-binding molecules can be used to screen expression libraries for variant polypeptides of the invention as described herein. They can also be used to detect polypeptides, fragments, variants and derivatives of the invention as described hereinafter.

5. Methods of Detection

5.1. Detection of Polypeptides According to the Invention

The invention also extends to a method of detecting in a sample a polypeptide, fragment, variant or derivative as broadly described above, comprising contacting the sample with an antigen-binding molecule as described in Section 4 or an IAP to which binds specifically the polypeptide, fragment, variant or derivative and detecting the presence of a complex comprising said polypeptide, fragment, variant or derivative and a member selected from said antigen-binding molecule and said IAP, in said contacted sample.

Although the detection methods below are discussed in the context of antigen-binding molecules, practitioners in the art will appreciate that such methods could also be adapted to IAP-based detection methods.

The formation of a complex comprising an antigen-binding molecule and a target polypeptide may be carried out using any suitable technique. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a biological fluid and preferably a tissue biopsy such cardiac or hepatic tissue.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. patent specifications U.S. Pat. Nos. 4,366,241, 4,843,000, and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable color change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

5.2. Detection of Polynucleotides According to the Invention

In another embodiment, the method for detection comprises detecting expression in a cell of a polynucleotide encoding said polypeptide, fragment, variant or derivative. Expression of the said polynucleotide may be determined using any suitable technique. For example, a labelled polynucleotide encoding a said member may be utilised as a probe in a Northern blot of a RNA extract obtained from the muscle cell. Preferably, a nucleic acid extract from the animal is utilised in concert with oligonucleotide primers corresponding to sense and antisense sequences of a polynucleotide encoding a said member, or flanking sequences thereof, in a nucleic acid amplification reaction such as RT PCR. A variety of automated solid-phase detection techniques are also appropriate. For example, very large scale immobilised primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art.

6. Screening for Modulators of DIABLO

The present invention resides in part in the discovery of a pro-apoptotic protein DIABLO that can directly interact with IAPs, which are suitably of mammalian or baculoviral origin, and interfere with IAP-mediated protection of apoptosis. Accordingly, it is believed that modulation of the level and/or functional activity of DIABLO, inclusive of fragments variants and derivatives of DIABLO, or modulation of expression of these molecules could modulate DIABLO-mediated cell death.

The invention therefore provides a method for screening for an agent which modulates cell death, comprising contacting a preparation comprising a polypeptide as broadly described above or a genetic sequence encoding said polypeptide with a test agent; and detecting a change in the level and/or functional activity of said polypeptide or an expression product of said genetic sequence.

Screening for modulatory agents according to the invention can be achieved by any suitable method. For example, the method may include contacting a cell, preferably a muscle cell, comprising a genetic sequence expressing a target protein such as DIABLO, with an agent suspected of having said modulatory activity and screening for the modulation of that protein, or the modulation of expression of the genetic sequence encoding that protein, or the modulation of the activity or expression of a downstream cellular target of said protein. Detecting such modulation can be achieved utilising techniques including, but not restricted to, Western blotting, ELISA, and RT-PCR.

It will be understood that a genetic sequence from which the target protein of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for the target protein or it may comprise a portion of that coding sequence (e.g. a domain such as a protein binding domain) or a portion that regulates expression of a product encoded by the polynucleotide (e.g., a promoter). For example, the promoter that is naturally associated with the genetic sequence may be introduced into the cell, which is the subject of testing. In this regard, where only the promoter is utilised, detecting modulation of the promoter activity can be achieved, for example, by operably linking the promoter to a suitable reporter polynucleotide including, but not restricted to, luciferase, β-galactosidase and CAT. Modulation of expression may be determined by measuring the activity associated with the reporter polynucleotide.

In another example, the subject of detection could be a downstream regulatory target of the target protein, rather than target protein itself or the reporter molecule operably linked to a promoter of a gene encoding a protein the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the genetic sequence encoding the target protein or expression product itself or which modulate the expression of an upstream molecule, which subsequently modulates the expression of the genetic sequence encoding the target protein or expression product activity. Accordingly, these methods provide a mechanism of detecting agents, which either directly or indirectly modulate the expression and/or activity of a target protein according to the invention.

In a series of preferred embodiments, the present invention provides assays for identifying small molecules or other compounds (i.e., modulatory agents), which are capable of inducing or inhibiting the expression of DIABLO or DIABLO-related genes and proteins. The assays may be performed in vitro using non-transformed cells, immortalised cell lines, or recombinant cell lines. In addition, the assays may detect the presence of increased or decreased expression of DIABLO or other DIABLO-related genes or proteins on the basis of increased or decreased mRNA expression (using, for example, the nucleic acid probes disclosed herein), increased or decreased levels of DIABLO or other DIABLO-related protein products (using, for example, the anti-DIABLO antigen-binding molecules disclosed herein), or increased or decreased levels of expression of a reporter gene (e.g., β-galactosidase or luciferase) operatively linked to a DIABLO 5′ regulatory region in a recombinant construct.

Thus, for example, one may culture cells known to express a particular DIABLO and add to the culture medium one or more test compounds. After allowing a sufficient period of time (e.g., 6-72 hours) for the compound to induce or inhibit the expression of the DIABLO, any change in levels of expression from an established baseline may be detected using any of the techniques described above and well known in the art. In particularly preferred embodiments, the cells are NT2 cells. Using the nucleic acid probes and/or antigen-binding molecules disclosed herein, detection of changes in the expression of a DIABLO, and thus identification of the compound as an inducer or repressor of DIABLO expression, requires only routine experimentation.

In particularly preferred embodiments, a recombinant assay is employed in which a reporter gene such a β-galactosidase or luciferase is operably linked to the 5′ regulatory regions of a DIABLO gene. Such regulatory regions may be easily isolated and cloned by one of ordinary skill in the art in light of the present disclosure of the coding regions of these genes. The reporter gene and regulatory regions are joined in-frame (or in each of the three possible reading frames) so that transcription and translation of the reporter gene may proceed under the control of the DIABLO regulatory elements. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high through-put assay for the identification of inducers and repressors of the DIABLO gene.

Compounds identified by this method will have potential utility in modifying the expression of DIABLO or other DIABLO-related genes in vivo. These compounds may be further tested in the animal models to identify those compounds having the most potent in vivo effects. In addition, as described above with respect to small molecules having DIABLO-binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit DIABLO activity is provided in which a purified preparation of DIABLO protein is combined with a candidate agent under conditions in which DIABLO is activated, and the level of DIABLO activity is measured by a suitable assay.

For example, a DIABLO inhibitor can be identified by measuring the ability of a candidate agent to decrease DIABLO activity in a cell (e.g., a muscle cell or a liver cell). In this method, a cell that is capable of expressing DIABLO is exposed to, or cultured in the presence and absence of, the candidate agent under conditions in which DIABLO is activated in the cell, and apoptosis detected. An agent tests positive if it inhibits or protects against apoptosis. For example, inhibition of DIABLO activity could be tested in NT2 cells as more fully described hereinafter (see for example FIGS. 7B and C). A molecule that interferes with the interaction between DIABLO and an IAP such as MIHA would allow survival of cells that are subjected to UV irradiation.

Alternatively, DIABLO-IAP (e.g., MIHA) interactions can be studied in the yeasts Saccharomyces cerevisiae and S. pombe. IAP interactions and screens for inhibitors can be carried out using the yeast Two-Hybrid system, which takes advantage of transcriptional factors that are composed of two physically separable, functional domains (Phizicky and Fields, 1994, Microbiol. Rev. 59(1): 94-123). The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and, if interactions occur, activation of a reporter gene (e.g., lacZ) produces a detectable phenotype. In the present case, for example, S. cerevisiae is co-transformed with a vector expressing a MIHA (or other IAP)-GAL4 activation domain fusion and a vector expressing a DIABLO-GAL4 binding domain fusion. If lacZ is used as the reporter gene, co-expression of the fusion proteins will produce a blue color. Small molecules or other candidate compounds which interfere with interaction of DIABLO and the IAP will result in loss of color of the cells. This system could be used to screen for small molecules that inhibit the DIABLO-IAP interaction and, hence, protect the yeast against cell death, and to determine the residues in IAP and DIABLO that are involved in their interaction. For example, reference may be made to the yeast Two-Hybrid systems as disclosed by Munder et al. (1999, Appl. Microbiol. Biotechnol. 52(3): 311-20) and by Young et al. (1998, Nat. Biotechnol. 16(10): 946-50), which are especially preferred to screen for small molecules (e.g., non-peptide drugs) that inhibit the DIABLO - IAP interaction. Molecules thus identified by this system could then be re-tested in mammalian cells.

Expression of caspase 3 causes death of S. pombe and S. cerevisiae, but this can be inhibited by co-expression of IAPs, including MIHA. In an alternate embodiment, for example, S. pombe is co-transformed with vectors expressing caspase 3, MIHA and DIABLO. Such yeast die because DIABLO prevents MIHA from inhibiting caspase 3. Small molecules or other candidate compounds or gene library products which interfere with interaction of DIABLO and the IAP will result in survival of the yeast, which can then be detected by colony growth.

In yet another embodiment, random peptide libraries consisting of all possible combinations of amino acids attached to a solid phase support may be used to identify peptides that are able to bind to DIABLO or to a functional domain thereof. Identification of molecules that are able to bind to DIABLO may be accomplished by screening a peptide library with a recombinant soluble DIABLO. The DIABLO may be purified, recombinantly expressed or synthesised by any suitable technique. A suitable purification procedure is disclosed for example hereinafter.

To identify and isolate the peptide/solid phase support that interacts and forms a complex with DIABLO, it is necessary to label or “tag” DIABLO. DIABLO may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with DIABLO, may be performed using techniques that are routine in the art. Alternatively, DIABLO expression vectors may be engineered to express a chimeric DIABLO containing an epitope for which an antigen-binding molecule exists. The epitope specific antigen-binding molecule may be tagged using methods well known in the art including labelling with enzymes, fluorescent dyes or colored or magnetic beads as for example described in Section 5.

The “tagged” DIABLO conjugate is incubated with the random peptide library for 30 minutes to one hour at 22° C. to allow complex formation between DIABLO and peptide species within the library. The library is then washed to remove any unbound DIABLO protein. If DIABLO has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4′,4′-diamnobenzidine (DAB), respectively. After incubating for several minutes, the peptide/solid phase-DIABLO complex changes color, and can be easily identified and isolated physically under a dissecting microscope with a micromanipulator. If a fluorescent-tagged DIABLO molecule has been used, complexes may be isolated by fluorescent activated sorting. If a chimeric DIABLO protein expressing a heterologous epitope has been used, detection of the peptide/DIABLO complex may be accomplished by using a labelled epitope specific antigen-binding molecule. Once isolated, the identity of the peptide attached to the solid phase support may be determined by peptide sequencing.

7. Method of Modulating Cell Death

The invention also features a method for modulating cell death, comprising contacting said cell with an agent for a time and under conditions sufficient to modulate the level and/or functional activity of a polypeptide as broadly described above. In a preferred embodiment, the agent decreases the level and/or functional activity of said polypeptide.

In a preferred embodiment, the agent is capable of decreasing the level and/or activity of a DIABLO. Any suitable DIABLO inhibitor may be used and, in this regard, suitable DIABLO inhibitors may be identified or produced by methods as for example disclosed in Section 6.

Alternatively, the DIABLO inhibitor may comprise oligoribonucleotide sequences, that include anti-sense RNA and DNA molecules and ribozymes that function to inhibit the translation of DIABLO-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a gene encoding a polypeptide according to the invention, are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of DIABLO RNA sequences.

Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of RNA molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

8. Methods of Treatment/prevention and Compositions Relating Thereto

The invention also encompasses a method for treatment and/or prophylaxis of a condition associated with expression or activation of DIABLO, said composition comprising an agent which reduces the level and/or functional activity of a polypeptide as broadly described above, together with a pharmaceutically acceptable carrier. The condition is preferably selected from any one or more of cancer, vascular disease, hepatic disease, autoimmune disease and neurodegenerative disease. In an especially preferred embodiment of this type, the condition is tissue damage, including muscle tissue damage associated with heart attack and hepatic tissue damage associated with a liver disease.

The modulatory agent can be administered to a patient either by itself, or in pharmaceutical compositions where it is mixed with suitable pharmaceutically acceptable carrier.

Depending on the specific conditions being treated, modulatory agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the condition, which is preferably heart damage resulting from a cardiac arrest or liver damage which may result for example from alcohol (e.g., cirrhosis) or viral-mediated disease (e.g., hepatitis). The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate tissue levels of a polypeptide, fragment, variant or derivative of the invention, and progression of the disorder. In any event, those of skill in the art may readily determine suitable dosages of the agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the modulatory agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or micro spheres.

Modulating agents of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition of DIABLO activity). Such information can be used to more accurately determine useful doses in humans.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p.1).

Dosage amount and interval may be adjusted individually to provide plasma levels of the active agent which are sufficient to maintain DIABLO-inhibitory effects or IAP stabilising effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably a heart muscle tissue or a liver tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In an alternate embodiment, a polynucleotide encoding a modulatory agent of the invention may be used as a therapeutic or prophylactic composition in the form of a “naked DNA” composition as is known in the art. For example, an expression vector comprising said polynucleotide operably linked to a regulatory polynucleotide (e.g. a promoter, transcriptional terminator, enhancer, etc.) may be introduced into an animal where it causes production of a modulatory agent in vivo, preferably in a heart muscle tissue or a liver tissue.

The step of introducing the expression vector into a target cell or tissue will differ depending on the intended use and species, and can involve one or more of non-viral and viral vectors, cationic liposomes, retroviruses, and adenoviruses such as, for example, described in Mulligan, R. C., (1993 Science 260: 926-932. Such methods can include, for example:

Local application of the expression vector by injection (Wolff et al., 1990, Science 247: 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.

General systemic delivery by injection of DNA, (Calabretta et al., 1993, Cancer Treat. Rev. 19: 169-179), or RNA, alone or in combination with liposomes (Zhu et al., 1993, Science 261: 209-212), viral capsids or nanoparticles (Bertling et al., 1991, Biotech. Appl. Biochem. 13: 390-405) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.

Injection or implantation or delivery by any means, of cells that have been modified ex vivo by transfection (for example, in the presence of calcium phosphate: Chen et al., 1987, Mole. Cell Biochem. 7: 2745-2752, or of cationic lipids and polyamines: Rose et al., 1991, BioTech. 10: 520-525), infection, injection, electroporation (Shigekawa et al., 1988, BioTech. 6: 742-751) or any other way so as to increase the expression of said polynucleotide in those cells. The modification can be mediated by plasmid, bacteriophage, cosmid, viral (such as adenoviral or retroviral; Mulligan, 1993, Science 260: 926-932; Miller, 1992, Nature 357: 455-460; Salmons et al., 1993, Hum. Gen. Ther. 4: 129-141) or other vectors, or other agents of modification such as liposomes (Zhu et al., 1993, Science 261: 209-212), viral capsids or nanoparticles (Bertling et al., 1991, Biotech. Appl. Biochem. 13 390-405), or any other mediator of modification. The use of cells as a delivery vehicle for genes or gene products has been described by Barr et al., 1991, Science 254: 1507-1512 and by Dhawan et al., 1991, Science 254 1509-1512. Treated cells can be delivered in combination with any nutrient, growth factor, matrix or other agent that will promote their survival in the treated subject.

In order that the invention may be readily understood and put into practical effect, particular preferred embodiments will now be described by way of the following non-limiting examples.

### EXAMPLES

### Example 1

### Transfections and Constructs

The human embryonic kidney carcinoma cell line 293T was grown in RPMI media supplemented with 10% FCS, 1% penicillin-streptomycin and 2% glutamine and was transiently transfected with different mammalian expression constructs using polyethyleneimine as described. (Boussif et al., 1995). pEF BOS expression constructs encoding C- and N-terminally Flag-epitope tagged MIHA (Flag-MIHA), C-terminally Flag tagged MIHB (Flag-MIHB) and MIHC (Flag-MIHC), N-terminally Flag-tagged OpIAP (Flag-OpIAP) and N-terminally Flag tagged CrmADQMD variant (Flag-DQMD) have been previously described (Uren et al, 1996, Ekert et al, 1999). A pcDNA3 vector (kindly provided by David Huang) construct encoding DIABLO with a Kozak initiation site and a C-terminal HA epitope tag was generated by PCR from IMAGE consortium clone 775927 (accession AA276162) (HA-DIABLO). A pEF BOS expression construct encoding untagged DIABLO (Unt-DIABLO) with a Kozak initiation site was also generated by PCR. The pcDNA3 expression constructs encoding unrelated HA tagged proteins FLN29 (HA-FLN29) and ZAP1 (HA-ZAP1) were kindly provided by Jun-ichi Nezu and Shingo Toji respectively. Transfection efficiency was visualised by co-transfection with a pEF BOS expression vector encoding green fluorescent protein. One to two days post transfection, cells were metabolically labelled with ³⁵S methionine in RPMI/10% FCS for a further 24 hr prior to lysis.

The neuronal cell line NT2 was grown in DMEM supplemented with 10% FCS, 1% penicillin-streptomycin and 2% glutamine. Stable NT2 lines were made using 1×10⁷ NT2 cells transfected with 10 μg plasmid linearised with FspI and using lipofectamine (Gibco-BRL). Forty-eight hours after transfection cells were split into 4 separate plates and selected with puromycin (Sigma) 8 μg/mL. Puromycin resistant colonies were selected and expression of the transgene was determined by flow cytometry using anti-Flag M2 antibody (Sigma) or anti-Bcl-2 antibody (DAKO-Bcl-2 124) as previously described (Ekert et al, 1999). Transient transfection of NT2 cells was done using 20 μL Effectene™ (QIAGEN) and 4 μg of plasmid in 100 mm. plates.

### Example 2

### Cell Death Assays

Loss of cell viability was determined using propidium iodide (PI) uptake or annexin V staining assessed by flow cytometry (Becton Dickinson). For PI staining, cells were harvested from the plate with trypsin, spun at 1500×g and resuspended in PBS supplemented with 2% FCS and PI (1 μg/mL) (Sigma). For annexin V staining, harvested cells were incubated with biotinylated annexin V (Kindly provided by Andreas Strasser) followed by incubation with Streptavidin-P.E. GFP positive cells were analysed by flow cytometry for annexin V staining. Microscopy was performed on an Olympus microscope equipped with Hoffman and fluorescence optics.

### Example 3

### Immunoprecipitations and Western Blot Analysis

Cells were lysed in a Triton X-100 based lysis buffer (10% Triton X-100, 10% glycerol, 150 mM NaCl, 20 mM Tris pH 7.5, 2 mM EDTA, 1 mM PMSF, 10 μg/mL aprotinin and 10 μg/mL leupeptin) for 1 hr, and the nuclear and cellular debris cleared by centrifugation. Immunoprecipitations were performed using Flag specific MAb M2 covalently coupled to agarose beads (Sigma, Australia) and anti-HA antibody HA. 11 (BABCO, Mannheim, Germany) plus protein G Sepharose™. The immunoprecipitates were washed 5 times in lysis buffer and proteins eluted with 100 mM glycine pH 3. Proteins were separated by SDS PAGE or two-dimensional immobilized-pH-gradient (IPG)/SDS-PAGE (Pharmacia, UK) as described Ji et al., 1997). Immunoprecipitates were examined by Western blot analysis or autoradiography following transfer of proteins to nitrocellulose membranes (Hybond C-Extra, Amersham, UK). MAbs used for Western blots were anti-Flag M2 (Sigma), anti-HA High Affinity 3F10 (Boehringer Mannheim, Mannheim, Germany), anti-cIAP1 (R&D systems), anti-cIAP2 (R&D Systems), anti-cytochrome C (7H8.2C12, Pharmingen, USA), anti-VDAC (31HL, Calbiochem), anti-calnexin (N-terminal specific, Stressgen, Canada) and anti-caspase 3 (Pharmingen). Proteins were visualized by ECL (Amersham, UK) following incubation of membranes with HRP-coupled secondary antibodies.

### Example 4

### Sucrose Gradient Fractionation

293T cells were transiently transfected with Flag-MIHA and HA-DIABLO. Forty-eight hr post-transfection the cells were washed twice in PBS and lysed on ice in a hypotonic buffer (20 mM HEPES, pH 7.4, 5 MM MgCl₂, 10 mM KCl, 1 mM EDTA, 1 mM EGTA, 10 mM Tris pH 7.4, 1 mM PMSF, 10 μg/mL aprotinin and 10 μg/mL leupeptin) for 20 min followed by passaging 25 times through a 27G needle. Unlysed cells and debris were removed by centrifugation at 250 g for 5 min. The extract was then centrifuged over a 50-10% sucrose gradient (in 20 mM HEPES, pH 7.4, 5 mM MgCl₂ 10 mM KCl, 1 mM EDTA and 1 mM EGTA) at 40 000 rpm for 20 hr at 4° C. in a Sw40 Ti rotor. Fractions were collected by puncturing the bottom of the tube and were analysed by Western blot with various antibodies.

### Example 5

### Protein Analysis and Mass Spectrometry

Proteins were separated by 2D immobilised-pH-gradient IPG)/SDS-PAGE as described Ji et al., (1997) and detected by staining of the gel with Coomassie Phast-gel Blue R (Pharmacia, UK). Protein spots of interest were excised, digested in situ with trypsin (Moritz et al., 1996), resolved by capillary column reversed-phase HPLC (Moritz and Simpson, 1992), and identified by collision-induced dissociation (CID) tandem mass spectrometry (MS/MS) using a quadrupole ion-trap mass spectrometer (Finnigan model LCQ) equipped with electrospray-ionisation (ESI) (Reid et al., 1998). Peptide amino acid sequences were determined by manual de novo interpretation of their b- and y-type production series (Roepstorff and Fohlman 1984).

### Example 6

### Northern Blot Analysis

Tissue specific expression of DIABLO was examined by hybridisation of a mouse tissue specific Northern blot of poly A+ RNA (Clontech) with a 720 bp ³²P-labelled partial cDNA probe encompassing the entire coding region for DIABLO. Equal RNA loading was confirmed by subsequent probing of the membrane with a β-Actin cDNA probe (Clontech).

### Example 7

### Identification of DIABLO

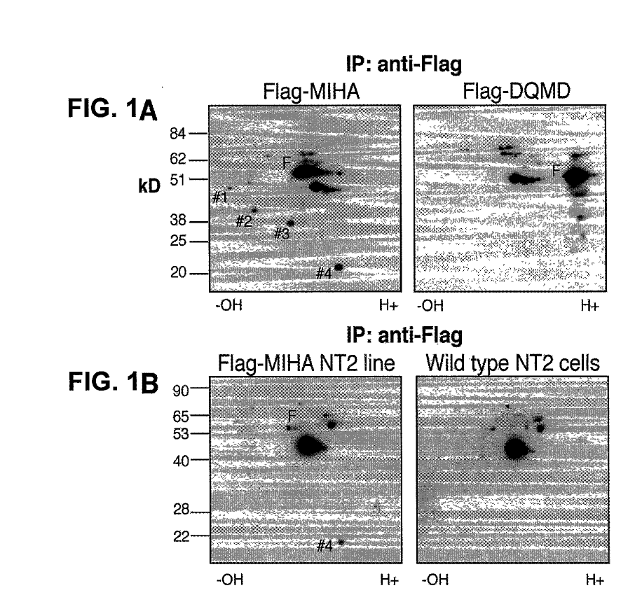

293T cells were transiently transfected with a cDNA encoding MIHA with a carboxy-terminal Flag epitope tag (Flag-MIHA) or a cDNA encoding control Flag-tagged CrmADQMD variant (Flag-DQMD, Ekert et al., 1999). Flag-MIHA and Flag-DQMD were immunoprecipitated from ³⁵S-labelled cell lysates with anti-Flag MAb-coupled agarose beads, and the immunoprecipitate examined by 2D IPG/SDS PAGE. Four proteins were observed co-immunoprecipitating with Flag-MIHA that were not observed in immunoprecipitates of the control Flag-tagged protein (FIG. 1). These four proteins also co-immunoprecipitated with MIHA bearing an amino-terminal Flag epitope tag transfected into 293T cells (data not shown). Furthermore, when experiments were carried out in neuronal NT2 cells stably expressing low levels of N-terminally Flag-tagged MIHA, although 3 of the proteins could no longer be immunoprecipitated, one, #4, a 23-kDa protein with a low pI of approximately 5.3, was still able to interact. This suggested that the binding between MIHA and protein #4 was physiological and unlikely to be a consequence of overexpression.

### Example 8

### Cloning of DIABLO

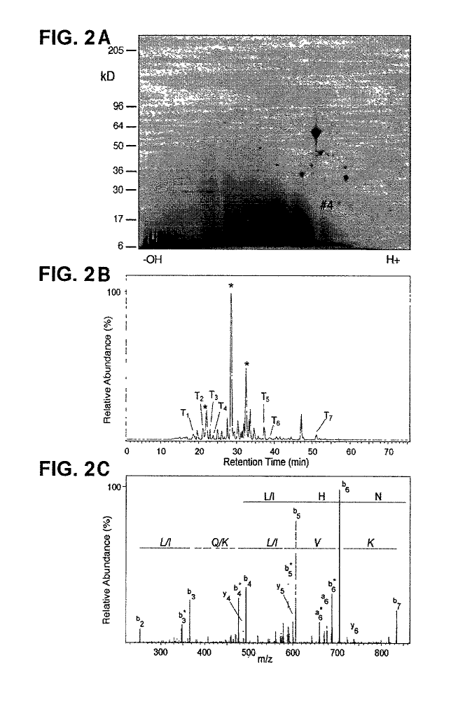

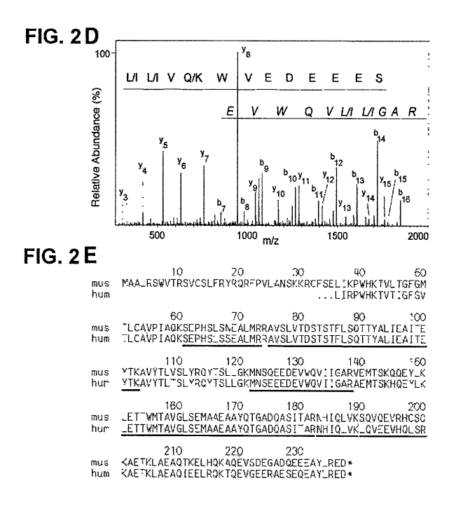

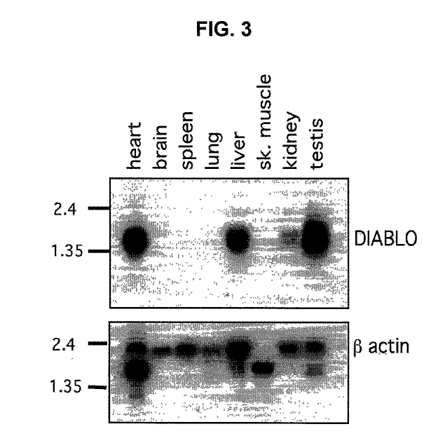

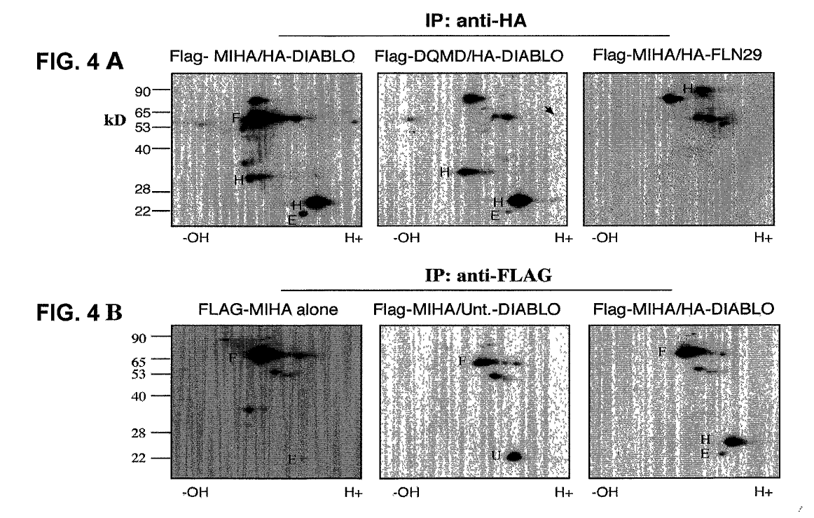

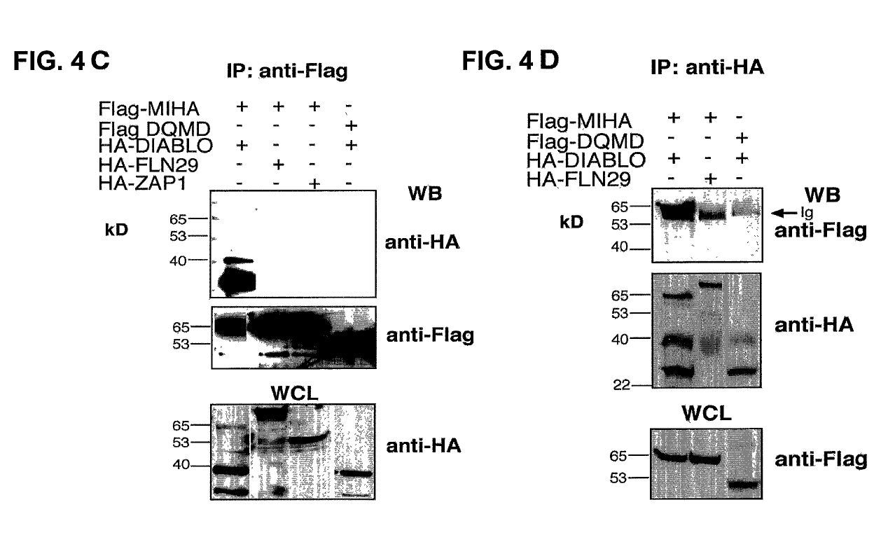

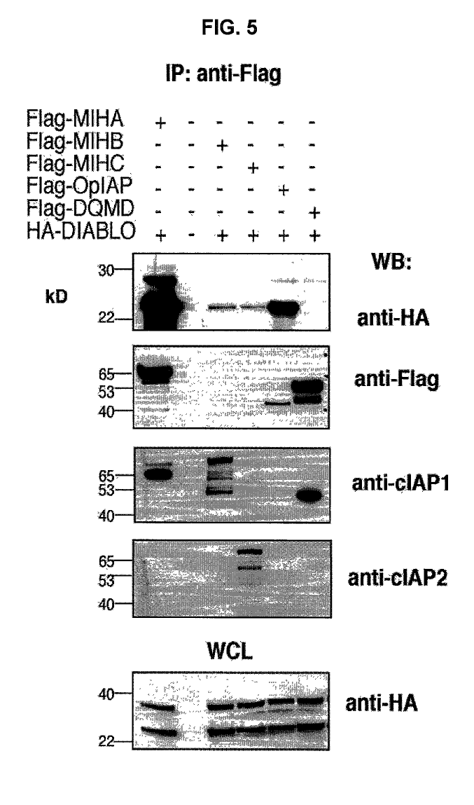

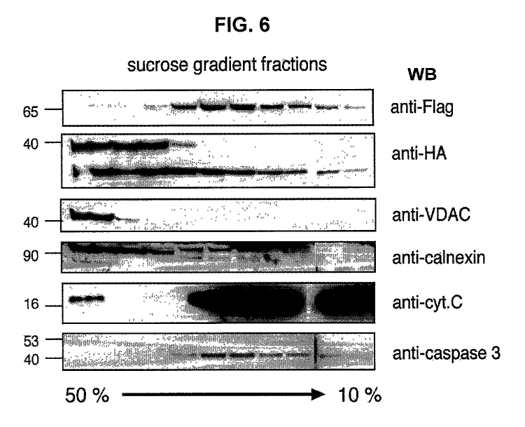

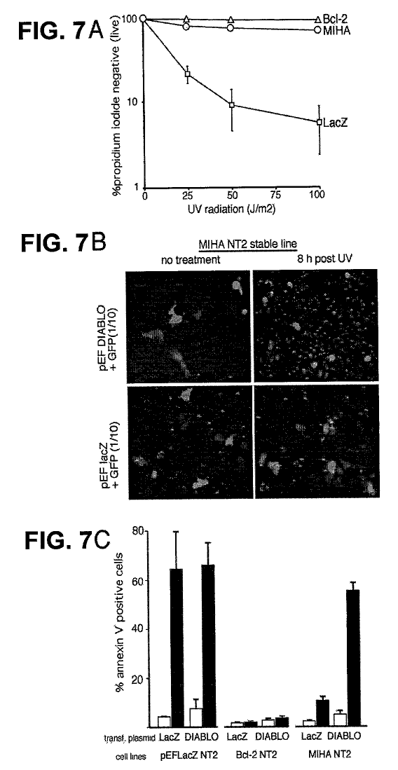

In order to identify protein #4, cellular lysates prepared from 100×15 cm petri dishes of 293T cells transiently transfected with Flag-MIHA were passed through a column of anti-Flag antibody-coupled agarose beads. After extensive washing, the bound proteins were eluted with acidic glycine, and separated by 2D IPG/SDS PAGE (FIG. 2A). A Coomassie stained gel spot corresponding to protein #4 was digested in situ with trypsin. The resultant peptides were extracted from the gel, subsequently resolved by capillary column RP-HPLC and sequenced by electrospray-ionisation tandem mass spectrometry (FIGS. 2B-E). Since automated database searching of the uninterpreted MS/MS spectra to identify the peptides was unsuccessful, manual de novo sequence analysis was employed to obtain either partial or complete amino acid sequence information on six peptides.

The peptide sequences recovered were used to search DNA EST databases using TFASTA and TBLASTN. Perfect agreement was found with both human and mouse sequences. The murine IMAGE consortium EST clone (accession AA276162) that extended most 5′ was obtained from Research Genetics and fully sequenced. This 1356 bp cDNA clone extended further 5′ than the human ESTs available and had an open reading frame encoding a 237-amino acid protein with predicted molecular weight of 26.8 kD and pI of 6.5. This is slightly larger and more basic than that of the isolated protein #4, suggesting post-translational modification. Although no upstream stop codon was present, two rat EST clones (AA686702, H35263) commencing at approximately the same site and with the same open reading frame were noted in the database. Further searches of the DNA databases using the full length mouse sequence also revealed a homologue in the Japanese flounder (ESTI 197472). No peptide motifs could be recognised using the PROSITE database.

### Example 9

### DIABLO Expression Pattern

Northern analysis of mRNA from adult mouse tissues showed that a 1.4 kb DIABLO message was most abundant in heart, liver, kidney and testis. Expression was not readily detected in skeletal muscle, lung, spleen or brain (FIG. 3). Nevertheless, human DIABLO ESTs derived from lung and cerebellum were noted in the database along with ESTs from kidney, heart, uterus and placenta.

### Example 10

### MIHA Interacts with DIABLO

To confirm that the cDNA obtained corresponded to MIHA interacting protein #4, we transfected 293T cells with cDNA encoding Flag-MIHA together with cDNA encoding putative murine protein (DIABLO) tagged on its carboxy terminus with a HA epitope (HA-DIABLO). Immunoprecipitations were prepared from ³⁵S methionine labelled cell extracts using either anti-Flag coupled agarose beads or anti-HA antibody plus protein G Sepharose, and were analysed by 2D IPG/SDS PAGE (FIGS. 4A and 4B). In the anti-HA immunoprecipitations, DIABLO was present in two forms, a 29-kDa protein with a pI of approximately 6.1 and a 24-kDa protein with a pI of approximately 5.4 (FIG. 4A). It is likely that the smaller protein represented a processed form of DIABLO from which the N-terminus had been cleaved. A protein with slightly lower molecular weight and slightly higher pI than the smaller of the HA-tagged spots was also be detected. This protein had the same mobility pattern as endogenous human protein spot #4, and was not recognised by anti-HA antibodies in Western analysis (not shown), and was therefore likely to be endogenous DIABLO. Because anti-HA antibodies were able to co-immunoprecipitate endogenous DIABLO along with transfected HA-tagged DIABLO, two or more DIABLO molecules must be able to form complexes in either the presence or absence of MIHA.

Flag-MIHA was clearly detected in the anti-HA immunoprecipitations from cells co-transfected with cDNAs encoding HA-tagged DIABLO and Flag-MIHA, whereas an unrelated Flag-tagged protein, Flag-DQMD, did not co-immunoprecipitate with HA-DIABLO. In addition, Flag-MIHA did not co-immunoprecipitate with an unrelated HA-tagged control. The high levels of ³⁵S labelled Flag-MIHA present in the immunoprecipitates of HA-DIABLO strongly suggest that there is a direct interaction between DIABLO and MIHA.

In immunoprecipitates of Flag-MIHA from cells transfected with Flag-MIHA alone, endogenous DIABLO is indicated (FIG. 4B). In Flag-MIHA immunoprecipitates from cells co-transfected with cDNA encoding untagged murine DIABLO a much stronger signal at this exact location was seen, presumably representing the co-expressed untagged murine DIABLO protein. In MIHA immunoprecipitates from cells co-transfected with MIHA and HA-tagged DIABLO, a protein was detected which migrated slightly above and with a more acidic pI than the endogenous molecule that was also visible. This slight shift in molecular weight and increased acidity can be accounted for by the HA tag. Interestingly, although DIABLO is present in the anti-HA immunoprecipitates in two forms, MIHA. interacts preferentially with the lower molecular weight, low pI, processed form.

The specificity of the DIABLO-MIHA interaction was also demonstrated by Western blot analysis. Flag-MIHA specifically co-immunoprecipitated HA-DIABLO but not two unrelated HA tagged proteins HA-FLN29 and HA-ZAP1 (FIG. 4C). HA-DIABLO was not co-immunoprecipitated with Flag-DQMD. In reverse, HA-DIABLO specifically co-immunoprecipitated Flag-MIHA but not Flag-DQMD and Flag-MIHA was not co-immunoprecipitated with control HAFLN29 (FIG. 4D).

### Example 11

### DIABLO Interacts with Several Anti-apoptotic IAPs

To test whether DIABLO is able to interact with other IAPs, we transfected 293T cells with cDNAs encoding Flag-tagged IAPs together with HA-DIABLO. The IAPs were inununoprecipitated with anti-Flag MAb-coupled agarose beads and the immunoprecipitates examined for the presence of DIABLO by Western analysis using anti-HA antibodies. As before, DIABLO was present in the whole cell lysate (WCL) in two forms, but MIHA interacted preferentially with the processed form, with only weak interaction with the non-processed protein being detectable. MIHB, MIHC and the baculoviral IAP from Orgyia pseudotsugata NPV, OpIAP, but not the control Flag-DQMD, were also able to interact with the processed form of DIABLO. Although less HA-DIABLO was detectable in these immunoprecipitates, these proteins were not very highly expressed in 293T cells. This was evident when the membrane was probed with anti-Flag MAb, where the signal for OpIAP was very weak compared to that of Flag-MIHA and Flag-DQMD, and Flag-MIHB and Flag-MIHC were not detectable. Flag-MIHB and Flag-MIHC could be detected following sequential probing of the membrane with the more sensitive cIAP 1 and cIAP2 specific Mabs respectively. These results indicate that despite low expression of MIHB and MIHC, there is a high stoichiometry of interaction between these IAPs and DIABLO.

### Example 12

### MIHA Shares a Cellular Compartment with Processed DIABLO

To confirm whether the interaction between IAPs and DIABLO could occur in vivo, we examined whether they reside in the same cellular compartment. 293T cells transiently expressing Flag-MIHA and HA-DIABLO were harvested and lysed in hypotonic buffer by passaging through a 27G needle (see Experimental Procedures). The extracts were then separated by centrifugation on 50%-10% sucrose gradients and the fractions analysed by Western blot using a series of antibodies (FIG. 6).

Membrane-associated proteins localised to the high percentage sucrose fractions, i.e. the bottom 3 to 4 fractions collected, as indicated by the presence of the mitochondrial membrane protein VDAC and the ER associated protein calnexin. Although small amounts of cytochrome C, a non-membrane associated mitochondrial protein, were also evident in these compartments, it was much more abundant in the cytosolic fractions, presumably due to mitochondrial leakage during extraction. Flag-MIHA was in the more buoyant cytosolic fractions and was virtually absent in membrane fractions, as was caspase 3. Interestingly, although the larger form of DIABLO was in the membrane associated fractions, N-terminally processed DIABLO, the form which preferentially interacts with MIHA, co-localised with MIHA. in the cytosol, in addition to being present in the membrane fractions.

### Example 13

### DIABLO is Able to Inhibit Protection of Cells by MIHA

To determine whether DIABLO was able to influence the anti-apoptotic activity of MIHA the inventors chose a system in which expression of MIHA is critical for cell survival. Parental NT2 cells undergo apoptosis when exposed to UV radiation, but clones that stably express MIHA do not (FIG. 7A). These cells were transfected with vectors encoding DIABLO and GFP, and analysed them by flow cytometry and fluorescence microscopy so that successfully transfected (green) could be analysed. When exposed to UV radiation, cells stably expressing MIHA transfected with pEFLacZ remained highly viable, whereas the DIABLO-transfected cells were killed (FIGS. 7B upper right panel and 7C MIHA-NT2 DIABLO). Sensitivity to UV radiation was not caused by transfection per se, because cells successfully transfected with a LacZ expression vector and the GFP construct rather than DIABLO and the GFP construct remained viable (FIG. 7A). Transfection of DIABLO did not cause apoptosis in NT2 cells stably expressing MIHA (FIG. 7B lower right panel) or Bcl-22 (data not shown), in the absence of UV radiation. Like MIHA, Bcl-2 is able to protect NT2 cells from apoptosis induced by UV radiation (FIG. 7A). However, DIABLO was not able to counter the protection against apoptosis afforded by Bcl2 (FIG. 7C).

### Discussion

Pro-apoptotic Drosophila proteins Grim, Reaper and HID appear to function by interacting with IAPs, disengaging them from caspases, and thereby allowing cell death to proceed (Wang et al., 1999). While Grim, Reaper and HID are required for normal development in the fly, no mammalian homologues of these proteins have been identified (Chen et al., 1996, Grether et al., 1995, White et al., 1994). However, it should be noted that DIABLO has no sequence similarity with any other protein previously described, but shares some functions with Grim, Reaper and HID. Like them, DIABLO interacts with IAPs, including MIHA, MIHB, MIHC and OpIAP, and interferes with the protective effect exerted by MIHA against UV radiation-induced cell death. Unlike Grim, Reaper and HID, however, DIABLO does not appear to induce apoptosis in healthy cells.

The inventors initially observed human DIABLO as a 23-kDa protein with a low isoelectric point (pI=5.3) which co-immunoprecipitated with MIHA. Full-length mouse DIABLO (29-kDa, pI=6. 1) undergoes N-terminal processing to yield the smaller form of the protein (23-kDa, pI=5.4) that preferentially interacted with IAPs. The higher molecular weight form was restricted to membrane fractions, while the processed, IAP-interacting form was present in both membrane fractions and the cytosol, where both MIHA and caspase 3 were found. The subcellular localisation of DIABLO may be significant if release of the IAP-binding, processed form from membranes is regulated following apoptotic stimuli.

DIABLO interacted with all the IAPs tested, including baculoviral OpIAP. While MIHA, MIHB and MIHC have all been shown to bind and directly inhibit caspases, direct inhibition of active caspases by OpIAP has not been described. For example, OpIAP protected insect cells from Reaper and UV induced cell death but not from cell death induced by expression of active caspases (Seshagiri et al., 1997). The interaction of OpIAP with DIABLO presents an alternative route for OpIAP mediated protection that does not involve direct caspase inhibition: OpIAP (and possibly other IAPs) may protect cells by sequestering cellular DIABLO, thus preventing it from disrupting the interaction of other IAPs with caspases. That DIABLO abrogated the protective effect exerted by MIHA against UV radiation-induced apoptosis but did not effect protection caused by Bcl-2 is consistent with Bcl-2 and MIHA regulating caspase activity independently.

Although DIABLO does not resemble Grim, Reaper or HID, it is clear that the pathways of apoptosis are similar between insects and mammals. In these organisms activity of the caspases is regulated by IAPs as well as by adaptor molecules such as Apaf-1 or DARK. Just as these adaptors are subject to upstream control, for example by Bcl-2 family members, IAPs appear to be regulated. DIABLO is the first mammalian protein to be identified that directly inhibits IAP function.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### Bibliography

Alnemri, E. S., Livingston, D. J., Nicholson, D. W., Salvesen, G., Thornberry, N. A., Wong, W. W and Yuan, J. (1996). Human ICE/CED-3 protease nomenclature. Cell 87,171.

Boldin, M. P., Goncharov, T. M., Goltsev, Y. V. and Wallach, D. (1996). Involvement of mach, a novel mortl/FADD-interacting protease, in Fas/APO-1- and TNF receptor-induced cell death. Cell 85, 803-815.

Boussif, O., Lezoualch, F., Zanta, M. A., Mergny, M. D., Scherman, D., Demeneix, B. and Behr, J. P. (1995). A versatile vector for gene and oligonucleotide transfer into cells in culture and in vivo - polyethylenimine. Proc. Natl. Acad. Sci. USA 92, 7297-7301.

Crook, N. E., Clem, R. J., and Miller, L. K. (1993). An apoptosis inhibiting baculovirus gene with a zinc finger like motif. J. Virol. 67, 2168-2174.

Deveraux, Q. L., Roy, N., Stennicke, H. R., Vanarsdale, T., Zhou, Q., Srinivasula, S. M., Alnemri, E. S., Salvesen, G. S. and Reed, J. C. (1998). IAPs block apoptotic events induced by caspase-8 and cytochrome c by direct inhibition of distinct caspases. EMBO J. 17, 2215-2223.

Deveraux, Q. L., Takahashi, R., Salvesen, G. S. and Reed, J. C. (1997). X-linked IAP is a direct inhibitor of cell-death proteases. Nature 388, 300-304.

Dorstyn, L., Colussi, P. A., Quinn, L. M., Richardson, H. and Kumar, S. (1999). DRONC, an ecdysone-inducible Drosophila caspase. Proc. Natl. Acad. Sci. USA 96, 4307-4312.

Duckett, C. S., Nava, V. E., Gedrich, R. W., Clem, R. J, Vandongen, J. L, Gilfillan, M. C., Shiels, H., Hardwick, J. M. and Thompson, C. B. (1996). A conserved family of cellular genes related to the baculovirus IAP gene and encoding apoptosis inhibitors. EMBO J. 15, 2685-2694.

Ekert, P. G., Silke, J. and Vaux, D. L. (1999). Inhibition of apoptosis and clonogenic survival of cells expressing crmA variants: optimal caspase substrates are not necessarily optimal inhibitors. EMBO J. 18, 330-338.

Hawkins, C. J., Uren, A. G., Hacker, G., Medcalf, R. L. and Vaux, D. L. (1996). Inhibition of interleukin 1-beta-converting enzyme-mediated apoptosis of mammalian cells by baculovirus IAP. Proc. Natl. Acad. Sci. USA 93, 13786-13790.

Hawkins, C. J., Wang, S. L. and Hay, B. A. (1999). A cloning method to identify caspases and their regulators in yeast: Identification of Drosophila IAP1 as an inhibitor of the Drosophila caspase DCP-1. Proc. Natl. Acad. Sci. USA 96, 2885-2890.

Hay, B. A., Wassarman, D. A. and Rubin, G. M. (1995). Drosophila homologs of baculovirus inhibitor of apoptosis proteins function to block cell death. Cell 83, 1253-1262.

Hu, Y. M., Benedict, M. A., Wu, D. Y., Inohara, N. and Nunez, G. (1998). Bcl-x-1 interacts with Apaf-1 and inhibits Apaf-1-dependent caspase-9 activation. Proc. Natl. Acad. Sci. USA 95, 4386-4391.

Irmler, M., Thome, M., Hahne, M., Schneider, R., Hoffmann, B., Steiner, V., Bodmer, J. L., Schroter, M., Burns, K., Mattmann, C., Rimoldi, D., French, L. E. and Tschopp, J. (1997). Inhibition of death receptor signals by cellular FLIP. Nature 388, 190-195.

Ji, H., Reid, G. E., Moritz, R. L., Eddes, L S., Burgess, A W. and Simpson, R. J. (1997). A two-dimensional gel database of human colon carcinoma proteins. Electrophoresis 18, 605613.

Kaiser, W. J., Vucic, D. and Miller, L. K. (1998). The Drosophila inhibitor of apoptosis D-IAP1 suppresses cell death induced by the caspase drICE. FEBS Letters 440, 243-248.

Liston, P., Roy, N., Tamai, K., Lefebvre, C., Baird, S., Chertonhorvat, G., Farahani, R., Mclean, M., Ikeda, J. E., Mackenzie, A. and Komeluk, R. G. (1996). Suppression of apoptosis in mammalian cells by NAIP and a related family of IAP genes. Nature 379, 349-353.

Moritz, R. L., Eddes, J. S., Reid, G. E. and Simpson, R. J. (1996). S-pyridylethylation of intact polacrylamide gels and in situ digestion of electrophoretically separated proteins: A rapid mass spectrometric method for identifying cysteine-containing peptides. Electrophoresis 17, 907-917.

Moritz, R. L. and Simpson, R. J. (1992). Application of capillary reversed-phase highperformance liquid chromatography to high-sensitivity protein sequence analysis. J. Chromatogr. 599, 119-130.

Muzio, M., Chinnaiyan, A. M., Kischkel, F. C., O'Rourke, K., Shevchenko, A., Ni, J., Scaffidi, C., Bretz, J. D., Zhang, M., Gentz, R., Mann, M., Krammer, P. H., Peter, M. E. and Dixit, V. M. (1996). FLICE, a novel FADD-homologous ICE/Ced-3-like protease, is recruited to the CD95 (Fas/APO-1) death-inducing signaling complex. Cell 85, 817-827.

Pan, G. H., O'Rourke, K. and Dixit, V. M. (1998). Caspase-9, Bcl-x-1, and Apaf-1 form a ternary complex. J. Biol. Chem. 273, 5841-5845.

Reid, G. E., Rasmussen, R. K., Dorow, D. S. and Simpson, R. J. (1998). Capillary column chromatography improves sample preparation for mass spectrometric: analysis: Complete characterisation of human α-enolase from two-dimensional gels following in situ proteolytic digestion. Electrophoresis 19, 946-955.

Roepstorff, P., and Fohlman, J. (1984). Proposal for a common nomenclature for sequence ions in mass spectra of peptides Biomedical Mass Spectrom. 11, 601-602.

Rothe, M., Pan, M. G., Henzel, W. J., Ayres, T. M. and Goeddel, D. V. (1995). The TNFR2TRAF signaling complex contains two novel proteins related to baculoviral-inhibitor of apoptosis proteins. Cell 83, 1243-1252.

Seshagiri, S. and Miller, L. K. (1997). Baculovirus inhibitors of apoptosis (IAPs) block activation of sf-caspase-1. Proc. Natl. Acad. Sci. USA 94, 13606-13611.

Uren, A. G., Pakusch, M., Hawkins, C. J., Puls, K. L. and Vaux, D. L. (1996). Cloning and expression of apoptosis inhibitory protein homologs that function to inhibit apoptosis and/or bind tumor necrosis factor receptor-associated factors. Proc. Natl. Acad. Sci. USA 93, 49744978.

Vaux, D. L. and Korsmeyer, S. J. (1999). Cell death in development. Cell 96, 245-254.

Wang, S. L., Hawkins, C. J., Yoo, S. J, Muller, H. A. J. and Hay, B. A. (1999). The Drosophila caspase inhibitor DIAP1 is essential for cell survival and is negatively regulated by HID. Cell 98, 453-463.

Zou, H., Henzel, W. J., Liu, X. S., Lutschg, A. and Wang, X. D. (1997). Apaf-1, a human protein homologous to C. elegans Ced-4, participates in cytochrome c-dependent activation of caspase-3. Cell 90, 405-413.

What is claimed is:

1. An isolated polypeptide comprising the sequence set forth in any one of SEQ ID NO:2 and 6, or a biologically active fragment thereof at least 8 amino acids in length.

2. The polypeptide of claim 1, comprising the sequence set forth in SEQ ID NO:2.

3. The polypeptide of claim 1, comprising the sequence set forth in SEQ ID NO:6.

4. The polypeptide of claim 1, wherein the fragment is selected from any one or more members in the group consisting of residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232 and 228-237 of SEQ ID NO:2.

5. An isolated polynucleotide comprising a nucleotide sequence encoding the sequence set forth in any one of SEQ ID NO:2 and 6, or a biologically active fragment thereof at least 8 amino acids in length.

6. The polynucleotide of claim 5, comprising a nucleotide sequence encoding the sequence set forth in SEQ ID NO:2.

7. The polynucleotide of claim 5, comprising a nucleotide sequence encoding the sequence set forth in SEQ ID NO:6.

8. The polynucleotide of claim 6, comprising the sequence set forth in SEQ ID NO:1.

9. The polynucleotide of claim 7, comprising the sequence set forth in SEQ ID NO:3.

10. A vector comprising the polynucleotide of claim 5.

11. An expression vector comprising the polynucleotide of claim 5 operably linked to a regulatory polynucleotide.

12. A host cell containing the vector of claim 10.

13. A host cell containing the vector of claim 11.

14. A method of producing a recombinant polypeptide comprising the sequence set forth in any one of SEQ ID NO:2 and 6, comprising:
culturing a host cell containing the vector of claim 10 such that said recombinant polypeptide is expressed from said polynucleotide; and
isolating said recombinant polypeptide.

15. A method of producing a recombinant polypeptide comprising the sequence set forth in any one of SEQ ID NO:2 and 6, comprising:
culturing a host cell containing the vector of claim 11 such that said recombinant polypeptide is expressed from said polynucleotide; and
isolating said recombinant polypeptide.

16. A method of producing a biologically active fragment of a polypeptide comprising the sequence set forth in any one of SEQ ID NO:2 and 6, comprising:
introducing a fragment of said polypeptide, or a polynucleotide from which the fragment can be expressed, into a cell; and
detecting cell death which is indicative of said fragment being a biologically active fragment.

17. A method of producing a biologically active fragment of a polypeptide comprising the sequence set forth in any one of SEQ ID NO:2 and 6, comprising:
contacting an IAP with a fragment of said polypeptide; and
detecting a reduction in activity of the IAP which is indicative of said fragment being a biologically active fragment.

18. A method of producing a variant of a parent polypeptide comprising the sequence set forth in any one of SEQ ID NO:2 and 6, comprising:
contacting an IAP with the modified polypeptide; and
detecting a reduction in activity of the IAP which is indicative of the modified polypeptide being said polypeptide variant.

19. A method of producing a polypeptide variant of a parent polypeptide comprising the sequence set forth in any one of SEQ ID NO:2 and 6, comprising:
introducing into a cell a modified polypeptide, whose sequence is distinguished from the parent polypeptide by substitution, deletion or addition of at least one amino acid, or a polynucleotide from which said modified polypeptide can be expressed;
detecting cell death which is indicative of the modified polypeptide being a said polypeptide variant.

20. A method of screening for an agent which modulates cell death, said method comprising:
contacting a preparation comprising a polypeptide comprising the sequence set forth in any one of SEQ ID NO:2 and 6, or a genetic sequence encoding said polypeptide with a test agent; and
detecting a change in the level and/or functional activity of said polypeptide or an expression product of said genetic sequence.

21. A method for detecting a specific polypeptide or polynucleotide sequence, comprising detecting a sequence of:
SEQ ID NO:2 or 6, or a fragment thereof at least 8 amino acids residues in length; or
SEQ ID NO:1 or 3, or a fragment thereof at least 24 nucleotides in length.

22. An antigen-binding molecule that specifically binds to a polypeptide comprising the sequence set forth in any one of SEQ ID NO:2 and 6, or a fragment thereof.

23. A method of detecting in a biological sample a polypeptide comprising the sequence set forth in any one of SEQ ID NO:2 and 6, comprising:
contacting the sample with an antigen-binding molecule that specifically binds to said polypeptide; and
detecting the presence of a complex comprising said antigen-binding molecule and said polypeptide in said contacted sample.

24. A method of detecting in a biological sample a polypeptide comprising the sequence set forth in any one of SEQ ID NO:2 and 6, comprising:
contacting the sample with an IAP which specifically binds to said polypeptide; and
detecting the presence of a complex comprising said IAP and said polypeptide, in said contacted sample.

25. A method for detecting a polypeptide comprising the sequence set forth in any one of SEQ ID NO:2 and 6, comprising:
detecting expression in a cell of a polynucleotide encoding said polypeptide.

26. A method for modulating the death of a cell, comprising:
contacting a cell with an agent, which modulates the level and/or functional activity of a polypeptide comprising the sequence set forth in any one of SEQ ID NO:2 and 6, for a time and under conditions sufficient to modulate the level and/or functional activity of said polypeptide.

27. The method of claim 26, wherein the agent decreases the level and/or functional activity of said polypeptide.

28. A composition for treatment and/or prophylaxis of a condition associated with expression or activation of DIABLO, said composition comprising an agent which reduces the level and/or functional activity of a polypeptide comprising the sequence set forth in any one of SEQ ID NO:2, and 6, together with a pharmaceutically acceptable carrier.

29. A method for treatment and/or prophylaxis of a condition associated with expression or activation of DIABLO, said method comprising administering to a patient in need of such treatment a therapeutically effective amount of an agent which modulates the level and/or functional activity of a polypeptide comprising the sequence set forth in any one of SEQ ID NO:2, 6 and 8, for a time and under conditions sufficient to modulate the level and/or functional activity of said polypeptide.

30. The method of claim 29, wherein said condition is selected from the group consisting of cancer, vascular disease, hepatic disease, autoimmune disease and neurodegenerative disease.

31. The method of claim 29, wherein said condition is tissue damage.

32. The method of claim 29, wherein said condition is muscle tissue damage associated with heart attack.

33. The method of claim 29, wherein said condition is hepatic tissue damage associated with a liver disease.
